(19) United States
(12) Patent Application Publication
(10) Pub. No.: US 2021/0190774 A1
(43) Pub. Date: Jun. 24, 2021

(54) COMPOSITIONS AND METHODS FOR THE DETECTION AND MOLECULAR PROFILING OF MEMBRANE BOUND VESICLES

(71) Applicant: The University of Memphis Research Foundation, Memphis, TN (US)

(72) Inventors: Xiaohua Huang, Memphis, TN (US); Allie Elyahb Kwizera, Memphis, TN (US); Vojtech Vinduska, Memphis, TN (US); Thang Ba Hoang, Memphis, TN (US)

(73) Assignee: The University of Memphis Research Foundation, Memphis, TN (US)

(21) Appl. No.: 16/954,112

(22) PCT Filed: Dec. 18, 2018
(86) PCT No.: PCT/US18/66251

§ 371 Date: Jun. 17, 2020

Related U.S. Application Data
(60) Provisional application No. 62/607,133, filed on Dec. 18, 2017.

(51) Int. Cl. G01N 33/543; G01N 33/553; G01N 33/552; G01N 21/65

(52) U.S. Cl. CPC G01N 33/54346; G01N 21/658; G01N 33/552; G01N 33/553

(57) ABSTRACT

The invention features compositions and methods related to the detection and molecular profiling of membrane bound vesicles using the Raman Extracellular Vesicle Assay (REVA). The method makes use of highly sensitive and specific surface enhanced Raman scattering technology to label and detect membrane bound vesicles that are captured on a miniaturized device based on the protein expression on the surface of the membrane bound vesicle.

### CROSS-REFERENCE TO RELATED APPLICATIONS

This application claims the benefit of and priority to the following U.S. Provisional Application Nos.: 62/607,133, filed Dec. 18, 2017, the entire contents of which are incorporated herein by reference.

### BACKGROUND OF THE INVENTION

Extracellular vesicles (EVs), including exosomes (EXOs) and microvesicles (MVs), have become a research subject of great excitement as a potential source of biomarkers in medicine. EVs are membrane bound vesicles, with EXOs derived from multivesicular bodies and MVs from plasma membrane. EVs carrying molecular constituents including proteins and nucleic acids of their originating cells represent an important mode of intercellular communication. A growing body of research has shown that cancer-derived EVs can transfer oncogenic activity and regulate angiogenesis, immunity, and metastasis to promote tumorigenesis and progression. EVs are present in various body fluids, such as blood, urine, saliva, and cerebrospinal fluids. Probing tumor-derived EVs in body fluids can therefore offer a non-invasive way to diagnose cancer, assess cancer progression, and monitor treatment responses.

The clinical use of EVs as cancer biomarkers has been limited by certain technical challenges. One such challenge is molecular detection and analysis of EVs. Due to their small size, EVs cannot be histologically examined using routine optical imaging, and they cannot be analyzed by traditional flow cytometry because of size limits (>200 nm). Western blot, enzyme-linked immunosorbent assays (ELISA), and mass spectrometry are commonly used to analyze EV proteins. These traditional approaches are impractical for longitudinal studies and clinical use because they are time-consuming, labor-intensive, and require relatively large amounts of samples. Despite recent advancements, technically simple, low cost, portable, rapid, efficient, sensitive, and specific technologies for EV molecular detection and surface protein analysis are needed.

### SUMMARY OF THE INVENTION

As described herein, the present invention features compositions and methods related to the detection and profiling of extracellular vesicles (e.g., exosomes, microvesicles) using the Raman Extracellular Vesicle Assay (REVA) that is technically simple, inexpensive, portable, rapid, efficient, highly sensitive, and highly specific. The method involves the use of highly sensitive and specific surface enhanced Raman scattering (SERS) nanotags (e.g. SERS gold nanorod (AuNR) tags) to detect and quantify surface proteins on membrane bound vesicles that are captured on a substrate (e.g., an array, Au-coated glass microscope slide, bead). The invention features the first application of SERS nanotags in the analysis of membrane bound vesicles from any biological sample (e.g., any cell or tissue, including body fluids, such as blood, urine, saliva, cerebrospinal fluid), or from any biological source (e.g., a human or non-human mammal).

In some embodiments, REVA is performed in at least two different ways, referred to herein as direct REVA (dREVA) and capture REVA (cREVA). In dREVA, EVs are immobilized on a lipophilic substrate, labeled with target-specific SERS nanotags (e.g. antibody-conjugated SERS AuNR tags), and detected with a portable Raman spectrometer. In cREVA, EVs are captured on target-specific substrate (e.g. antibody-conjugated Au-coated glass microscope slide), labeled with SERS nanotags (e.g. SERS AuNRs), and detected with a portable Raman spectrometer.

Some aspects of the present disclosure provide a lipophilic substrate comprising an amphiphilic polymer having a thiolated hydrophilic portion and a hydrophobic tail covalently bound to a silver or gold film, wherein the film is fixed to a solid support. In some aspects, a lipophilic substrate is provided that comprises 1,2-distearoyl-sn-glycero-3-phosphoethanolamine conjugated polyethylene glycol thiol (DSPE-PEG-SH) and 11-mercaptoundecyl tetra (ethylene glycol) (MU-TEG) covalently bound to a gold film, wherein the film is fixed to a solid support. In some embodiments, the solid support can be a microscope slide, membrane, or wafer. In some embodiments, the film is optically transparent or opaque, and in some embodiments, the film is gold or silver.

In some aspects, an array device is provided comprising a planar substrate that has an amphiphilic polymer containing a thiolated hydrophilic portion and a hydrophobic tail covalently bound to a film. The film is fixed to a planar support, and a flexible array interface contacts the planar substrate. The interface comprises a plurality of holes. A rigid array template comprising a plurality of holes is in contact with the interface, and the holes of the interface and the holes of the array are aligned.

Provided herein is an array device comprising a planar substrate comprising 1,2-distearoyl-sn-glycero-3-phosphoethanolamine conjugated polyethylene glycol thiol (DSPE-PEG-SH) and 11-mercaptoundecyl tetra (ethylene glycol) (MU-TEG) that is covalently bound to gold film. The film is fixed to the planar substrate, which is in contact with a flexible array interface that comprises a plurality of holes. A rigid array template in contact with the interface also comprises a plurality of holes, and the holes of the interface and the holes of the array are aligned. In some embodiments, the planar substrate is a glass plate or silicon wafer, and in some embodiments, the flexible array interface comprises rubber or silicone. In some embodiments the rigid array template comprises plastic or resin.

The array device comprises wells, wherein each well is at least 1 mm in diameter and the inter-well distance is at least 0.5 mm. The substrate, interface, and template are arranged to form fluid-tight wells.

Also provided herein are surface-enhanced Raman scattering nanotags. The nanotag comprising a plasmonic nanoparticle, a 16-mercaptohexadecanoic acid-linked polyethylene glycol covalently bound at the thiol terminal to a surface of the nanoparticle, an antibody bound to the PEG thiol with the thiol terminal bound to a surface of the nanoparticle, and a Raman reporter that is incorporated into the MHDA pocket on the surface of the nanoparticle. In some embodiments, the Raman reporter is an organic or inorganic dye, and in some embodiments the organic dye is selected from QSY21, IR820, IR783, BHQ, QXL 680, and DTTC. The inorganic dye may be a pyridine or aminothiophenol. In some embodiments, the Raman reporter is QSY21.

In some embodiments, the nanoparticle is gold or silver. In some embodiments, the nanoparticle is a core-shell nanoparticle. The core-shell nanoparticle can be a magnetic-metallic core-shell nanoparticle.

In some embodiments, the Raman reporter that is incorporated into the MHDA pocket is on the surface of a carbon nanosphere or nanotube. The nanoparticle is a gold or silver nanorod in some embodiments and can be between 10 nm and 100 nm.

Another aspect of the present disclosure provides a surface-enhanced Raman scattering nanotag comprising a plasmonic nanoparticle, a Raman reporter and a cetyltrimethylammonium bromide (CTAB) bilayer. And in some aspects, a surface-enhanced Raman scattering nanotag is provided that comprises a plasmonic nanoparticle, a Raman reporter and cetyltrimethylammonium bromide (CTAB) bilayer. The Raman reporter in some embodiments is QSY21.

Methods are also provided in the present disclosure. For example, one aspect provides a method for producing a target-specific capture array, the method comprising providing a device comprising a planar substrate comprising an amphiphilic polymer containing a thiolated hydrophilic segment and a hydrophobic tail covalently bound to a film. The film is fixed to the planar support. A flexible array interface is in contact with the planar substrate, and the interface comprises a plurality of holes. A rigid array template is in contact with the interface, and the rigid array comprises a plurality of holes. The holes of the interface and the holes of the array are aligned, thereby forming a well. Lastly, the method comprises depositing a target-specific capture molecule into each well of the array, thereby forming a capture array. In some embodiments, the capture molecule is an antibody, a single-chain antibody, a nanobody, or an aptamer, and the capture molecule specifically binds an antigen of interest.

Methods are also provided for producing an array device comprising a plurality of cells or membrane bound vesicles, the method comprising providing an array device comprising a planar substrate comprising 1,2-distearoyl-sn-glycero-3-phosphoethanolamine conjugated polyethylene glycol thiol (DSPE-PEG-SH) and 11-mercaptoundecyl tetra (ethylene glycol) (MU-TEG) covalently bound to a gold film in each well. The film is fixed to the planar substrate, and a flexible array interface is in contact with the planar substrate, wherein the interface comprises a plurality of holes. A rigid array template is in contact with the interface, and the rigid array comprises a plurality of holes. The holes of the interface and the holes of the array are aligned thereby forming a well. A cell or membrane bound vesicle is deposited into each well of the array device. In some embodiments, the cell is a cancer cell, blood cell, bacterial cell, epithelial cell, or a parasitic cell. In some embodiments, the membrane bound vesicle is an exosome, microvesicle, an oncosome, microsome, or cellular organelle. Some aspects of the present disclosure contemplate an array device comprising a cell or membrane bound vesicle produced as described supra.

A method is also provided for characterizing biomarkers on a plurality of cells or membrane bound vesicles, the method comprising contacting an array device with a nanotag of claim and detecting a biomarker present on the cell or membrane bound vesicle using Raman spectroscopy. The membrane bound vesicle is an exosome, microvesicles, oncosome, microsome, or cellular organelle.

Other aspects provide a method for characterizing biomarkers on a plurality of cells or membrane bound vesicles, the method comprising contacting the array device described supra with a sample comprising a cell or membrane bound vesicle under conditions suitable for binding. The bound cell or membrane bound vesicle is contacted with a nanotag, and a biomarker present on the cell or membrane bound vesicle is detected using Raman spectroscopy. In some embodiments, the membrane bound vesicle is an exosome, microvesicles, oncosome, microsome, or cellular organelle.

The present disclosure also provides methods for characterizing disease in a subject, the method comprising obtaining a biological sample comprises an extracellular vesicle from the subject and contacting a lipophilic substrate or an array device as disclosed herein with the biological sample under conditions suitable for binding a cell or membrane bound vesicle to the substrate or array device. The bound extracellular vesicle is contacted with a nanotag, and a biomarker present on the cell or membrane bound vesicle is detected using Raman spectroscopy.

A method is also provided for characterizing a disease in a subject, the method comprising obtaining a biological sample from the subject, wherein the sample comprises an extracellular vesicle. The array device is contacted with the biological sample under conditions suitable for binding the extracellular vesicle to the array device, and the bound extracellular vesicle is contacted with a nanotag. A biomarker present on the membrane bound vesicle is detected using Raman spectroscopy. In some embodiments, the biological sample is cell culture media, urine, blood, serum, plasma, cerebral spinal fluid, saliva, or ascites.

A method for characterizing biomarkers on a single membrane bound vesicle is also provide as an aspect of the present disclosure. The method comprises contacting a membrane bound vesicle with a nanotag and exposing the membrane bound vesicle to a light source. A dark field image is acquired of the membrane bound vesicle, and this image serves as a mask to localize the membrane bound vesicle. The localized membrane bound vesicle to a wavelength sufficient to elicit a signal from the nanotag and the brightness of the signal from the nanotag is detected. The presence or absence of a biomarker on the membrane bound vesicle is detected using Raman spectroscopy, thereby characterizing the exosome. In some embodiments, the membrane bound vesicle is an exosome. In some embodiments, the method further comprises contacting the exosome with an antibody that specifically binds an antigen associated with an exosome. In some embodiments, the antibody is conjugated to a polyethylene glycol thiol (PEG-SH) moiety, and the thiol portion of the PEG-SH moiety of the antibody is bound to a functionalized surface of an array or particle. The array, in some embodiments, has multiple wells comprising the bound antibody that specifically binds an antigen associated with an exosome. And in some embodiments of the present method, the array is contacted with a sample comprising exosomes, the exosomes are captured in the array wells.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “alteration” is meant a change (increase or decrease) in an analyte as detected by methods such as those described herein. In one embodiment, the alteration is in the level of a protein biomarker present on a membrane bound vesicle. As used herein, an alteration includes a 10% change in expression levels, preferably a 25% change, more preferably a 40% change, and most preferably a 50% or greater change in expression levels.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to characterizing the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Exemplary diseases that can be evaluated using a method of the invention include, but are not limited to, cancer and neurodegenerative diseases.

By “extracellular vesicle” is meant a membrane bound vesicle that is present extracellularly. Exemplary extracellular vesicles include exosomes and microvesicles.

By “marker” is meant any protein or polynucleotide having an alteration in expression level or activity that is associated with a disease or disorder.

By “mask” is meant an image that only includes pixels that match certain criteria, and subsequent analysis can be directed only to those areas on the mask image. By “membrane bound vesicle” (MBV or MBVs) is meant any vesicle comprising a membrane structure. Exemplary membrane bound vesicles include, but are not limited to, extracellular vesicles (EV or EVs), microvesicles (MV or MVs), exosomes, and apoptotic bodies.

By “Raman Spectroscopy” is meant the spectroscopic technique used to observe vibrational, rotational, and other low-frequency modes in a system. Raman spectroscopy has been commonly used in chemistry to provide a structural fingerprint by which molecules are identified.

By “surface enhanced Raman scattering spectroscopy” is meant the spectroscopic technique in which the Raman scattering signals of a small organic molecule, such as an organic dye, are enhanced by a plasmonic nanoparticle when the small organic molecule is present on, or close to, the surface of the plasmonic nanoparticle. Surface enhanced Raman scattering spectroscopy has been commonly used in chemistry to provide a structural fingerprint of the small organic molecules, or to detect a target with the use of surface enhanced Raman scattering nanotags.

By “surface enhanced Raman scattering nanotags” is meant a plasmonic nanoparticle coated with a Raman reporter. In one embodiment, the plasmonic nanoparticle is a silver or gold nanoparticles surrounded by a metal oxide shell containing a fluorophore. Surface enhanced Raman scattering nanotags provide for the detection and quantification of a target of interest via specific binding of the surface enhanced Raman scattering nanotags and the target of interest. This allows a unique “fingerprint” to be generated that includes the signals of the Raman reporters present on the target.

By “reduces” is meant a negative alteration of at least 10%, 25%, 50%, 75%, or 100%.

By “reference” is meant a standard or control condition.

By “subject” is meant a mammal, including, but not limited to, a human or non-human mammal, such as a bovine, equine, canine, ovine, or feline.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1A, FIG. 1B, and FIG. 1C show schematic illustrations of the methodology of direct Raman Extracellular Vesicle Assay (dREVA). FIG. 1A is a schematic diagram of the assay. FIG. 1B is an illustration showing the interaction of an EV with a lipophilic gold (Au) slide that allows for the immobilization of the EV on the slide. FIG. 1C is a schematic diagram showing the procedures if of dREVA. The assay contains four sequential steps: (1) Lipophilic surface modification of the Au slide; (2) EV immobilization; (3) EV labeling with the target-specific SERS AuNR tags; and (4) Signal collection with a Raman spectrometer. The EV device has multiple wells that allow for analysis of different proteins or different EVs on the same device simultaneously.

FIG. 2A, FIG. 2B, FIG. 2C, FIG. 2D, FIG. 2E, FIG. 2F, FIG. 2G, and FIG. 2H are schematic graphs and images showing how the EV array device was developed by assembly of a plastic array template, a rubber array interface, and the lipophilic Au slide. FIG. 2A is a schematic diagram of the fabrication of a Au slide with using the magnetron sputting technique on a standard glass microscope slide. FIG. 2B is a schematic of the fabrication of an EV array device. FIG. 2C is a schematic image of the top view of the EV array device showing the dimensions of the array and device. FIG. 2D, FIG. 2E, FIG. 2F, FIG. 2G, and FIG. 2H are photographic images showing the EV array device components, including the plastic array template (FIG. 2D), rubber array interface (FIG. 2E), and the Au slide (FIG. 2F). The dimensions of the plastic array tempate were 75×25×5 mm (L×W×H) with 56 wells (3 mm in diameter for each hole). The inter-well distance was 2 mm. The dimensions of the rubber array interface were 75×25×1.6 mm (L×W×H) with 56 wells (3 mm in diameter for each hole). The inter-well distance was 2 mm. The dimensions of the Au slide were 75×25×1 mm (L×W×H), and the glass microscope slide was coated with a 10-nm thick Au film. A photographic picture of the side view of the EV array device is shown in FIG. 2G and a photographic picture of the top view of the EV array device is shown in FIG. 2H.

FIG. 3 is a schematic diagram of the fabrication of the lipophilic Au slide, which includes sequential chemical modification of 1,2-distearoyl-sn-glycero-3-phosphoethanolamine conjugated polyethylene glycol thiol (DSPE-PEG-SH, long chain) and 11-mercaptoundecyl tetra (ethylene glycol) (MU-TEG, short chain).

FIG. 4A and FIG. 4B are schematic diagrams that depict the preparation and structures of target-specific SERS AuNR tags. FIG. 4A is schematic diagram of the preparation of antibody-conjugated SERS AuNRs that involves sequential binding of an antibody linked with a thiolated polyethylene glycol (PEG-SH) (represented as HS-PEG-Ab), adsorption of Raman reporter QSY21, and covalent binding with a protective polyethylene glycol (PEG) onto the as-synthesized AuNR, which is capped with a cetyltrimethylammonium bromide (CTAB) bilayer. FIG. 4B shows four chemical structure drawings: (top) the Raman reporter QSY21 that is a non-fluorescent organic dye; (second from top) an antibody with a thiolated polyethylene glycol (PEG-SH) linker (represented as HS-PEG-Ab in FIG. 4A); (second from bottom) a thiolated methoxy-PEG (HS-mPEG); and (bottom) a 16-mercaptohexadecanoic acid-linked PEG-SH (MHDA-PEG).

FIG. 5A, FIG. 5B, and FIG. 5C are graphs and images showing the characterization and target-specific capture of exosomes (EXOs) on the lipophilic Au slide. FIG. 5A shows the size distribution of EXOs derived from breast cancer MDA-MB-231 (MM231) cells (represented as MM231 EXOs) as measured by nanoparticle tracking analysis (NTA). The size for MM231 EXOs was 168±49 nm (mean±standard deviation). FIG. 5B shows a fluorescence image of MM231 EXOs that are immobilized on the DSPE-PEG-SH and MU-TEG modified Au slide. FIG. 5C shows a fluorescence image of MM231 EXOs that are immobilized on a MU-TEG modified Au slide. The results showed that EXOs were immobilized on the Au slide modified with DSPE-PEG-SH/MU-TEG but not with MU-TEG only.

FIG. 6A, FIG. 6B, and FIG. 6C are graghs and images characterizing AuNRs. FIG. 6A is a transmission electron microscopy (TEM) image of the AuNRs. The AuNRs are average 35 nm in length and 12 nm in width. FIG. 6B is a graph that shows the size distribution of AuNRs as measured by dynamic light scattering (DLS). The AuNRs have a mean hydrodynamic size of 38 nm. FIG. 6C is a graph that shows the absorption spectrum of AuNRs as measured by absorption spectroscopy. The AuNRs have a localized surface plasmon resonance (LSPR) at 720 nm.

FIG. 7A and FIG. 7B are graphs characterizing the SERS AuNRs tags and their stability. FIG. 7A is a graph that shows a typical SERS spectrum of 0.1 M SERS AuNR tags using QSY21 as the Raman reporter. FIG. 7B is a graph showing a comparison of the stability of the SERS AuNR tags between mPEG-SH and MHDA-PEG stabilizers. The MHDA-PEG has a remarkably improved stability of SERS AuNRs compared to the conventional mPEG-SH stabilizer.

FIG. 8 is an image showing a commercial high-performance Raman detection system (TSI ProRaman-L high performance spectrometer). The Raman system features a 785 nm laser wavelength, 200 μm Raman beam spot at focus, an integration time per spectrum larger than 50 ms, and a maximal laser power of 250 mW. Baseline correction is incorporated into the EZRaman Reader V88.1.8 MW software, and thus, the as-acquired spectrum is ready for use for data analysis.

FIG. 9A and FIG. 9B are graphs showing the specificity (FIG. 9A) and sensitivity (FIG. 9B) of dREVA. FIG. 9A is a graph showing the SERS spectra from different experiments. The experimental numbers (right side, FIG. 9A) represent experiments in which: (1) the Au slide was modified with MU-TEG and incubated with SERS AuNR-CD63 antibody; (2) the Au slide was modified with MU-TEG, incubated with MM231 EXOs, and incubated with SERS AuNR-CD63 antibody; (3) the Au slide was modified with MU-TEG and DSPE-PEG-SH, incubated with SERS AuNR-CD63 antibody; (4) the Au slide was modified with MU-TEG and DSPE-PEG-SH, incubated with MM231 EXOs, and incubated with SERS AuNR-CD63 antibody; (5) the Au slide was modified with MU-TEG and DSPE-PEG-SH, incubated with MM231 EXOs, and incubated with SERS AuNR-IgG protein; and (6) the Au slide was modified with MU-TEG and DSPE-PEG-SH, and incubated with MM231 EXOs. While experiment (4) showed strong signals, other treatments showed negligible signals suggesting high specificity of dREVA. FIG. 9B shows SERS signal intensity of the 1497 cm⁻¹ representative peak at different MM231 EXO concentrations. The data is presented as mean±standard deviation (n=3). The limit of detection (LOD) was determined to be 1×10⁶ EXOs/mL.

FIG. 10A, FIG. 10B, FIG. 10C, and FIG. 10D are graphs showing the detection and protein profiling of MM231 EXOs using dREVA with validation by the traditional enzyme-linked immunosorbent assay (ELISA). FIG. 10A shows the averaged SERS spectra (n=3) targeting of different surface proteins (EpCAM, CD44, HER2, CD81, CD63, and CD9) on MM231 EXOs. FIG. 10B is a graph showing the expression profile of the target proteins on MM231 EXOs based on the intensity values of the 1497 cm⁻¹ peak in the SERS spectra shown in FIG. 10A. FIG. 10C shows the expression profile of the target proteins on MM231 EXOs measured with ELISA. FIG. 10D shows the correlation of dREVA and ELISA. The results show that the two methods are highly correlated, with correlation coefficient of R²=0.99. The results show that MM231 EXOs have high expression of CD44 marker and low expression of EpCAM and HER2 markers. The EXOs are positive for all three EXO markers (CD81, CD63, and CD9).

FIG. 11A, FIG. 11B, FIG. 11C, and FIG. 11D are graphs showing flow cytometry analysis of the expression of surface markers on MM231 cells with IgG control. FIG. 11A shows the distribution of the fluorescence signals and scattering signals from MM231 cells labeled with phycoerythrin (PE)-conjugated EpCAM antibodies in terms of cell counts. FIG. 11B shows the distribution of the fluorescence signals and scattering signals from MM231 cells labeled with PE-conjugated CD44 antibodies in terms of cell counts. FIG. 11C shows the distribution of the fluorescence signals and scattering signals from MM231 cells labeled with PE-conjugated HER2 antibodies in terms of cell counts. FIG. 11D shows the distribution of the fluorescence signals and scattering signals from MM231 cells labeled with PE-conjugated IgG control proteins. The results show that MM231 cells have a high expression of the CD44 marker and a low expression of EpCAM and HER2 markers.

FIG. 12A, FIG. 12B, and FIG. 12C are schematic images showing the methodology of capture Raman Extracellular Vesicle Assay (cREVA). FIG. 12A is an image showing the principle of the array. FIG. 12B is an image showing the electrostatic interaction between SERS nanotags (e.g. AuNR tags) and the lipid membrane of EV that bases the labeling of EVs with the SERS nanotags. FIG. 12C is an image showing the procedures when using the assay for analyzing multiple samples. The assay contains four sequential steps: (1) Antibody functionalization of the Au slide; (2) EV binding; (3) EV labeling with SERS AuNR tags; and (4) Signal collection with a Raman spectrometer. The EV device has multiple wells that allow for analysis of different proteins or different EVs on the same device simultaneously.

FIG. 13 is an image showing the preparation of the capture Au slide, which includes sequential chemical modification with HS-PEG-Ab and MU-TEG.

FIG. 14 is an image showing the preparation of SERS AuNR tags via nonspecific adsorption of the QSY21 Raman reporters with the CTAB-capped AuNRs.

FIG. 15A and FIG. 15B are images showing target-specific capture of EXOs of the antibody functionalized Au slide. FIG. 15A shows a fluorescence image of MM231 EXOs that are captured using CD63 antibodies. FIG. 15B shows a fluorescence image of MM231 EXOs that are captured using IgG control. The results showed that EXOs were only captured on the slide modified with CD63 antibodies.

FIG. 16A and FIG. 16B are graphs showing the specificity (FIG. 16A) and sensitivity (FIG. 16B) of cREVA. FIG. 16A is a graph showing the SERS spectra from samples under different experiments. The experiment numbers on the right side of the graph represent an experiment in which: (1) the Au slide was modified with MU-TEG and incubated with SERS AuNR tags; (2) the Au slide was modified with MU-TEG, incubated with MM231 EXOs, and incubated with SERS AuNR tags; (3) the Au slide was modified with MU-TEG and CD63 antibodies and incubated with SERS AuNR tags; (4) the Au slide was modified with MU-TEG and CD63 antibodies, incubated with MM231 EXOs, and incubated with SERS AuNR tags; (5) the Au slide was modified with MU-TEG and IgG proteins, incubated with MM231 EXOs, and incubated with SERS Au NR tags. While experiment (4) showed strong signals, other treatments showed negligible signals, suggesting high specificity of cREVA. FIG. 16B shows SERS signal intensity of the 1497 cm⁻¹ representative peak at different EXO concentrations. The data is presented as mean±standard deviation (n=3). The LOD was determined to be 2×10⁶ EXOs/mL.

FIG. 17A, FIG. 17B, FIG. 17C, and FIG. 17D are graphs showing the detection and protein profiling of MM231 EXOs using cREVA with validation by ELISA. FIG. 17A shows SERS spectra targeting different surface proteins (EpCAM, CD44, HER2, CD1, CD63, and CD9) on MM231 EXOs. FIG. 17B shows the expression profile of the target proteins on MM231 EXOs based on the intensity values of the 1497 cm⁻¹ peak in the SERS spectra shown in FIG. 17A. FIG. 17C shows the expression profile of the target proteins on MM231 EXOs measured with ELISA. FIG. 17D shows the correlation of cREVA and ELISA. The results show that the two methods are highly correlated, with a correlation coefficient of R²=0.96. The results also show that MM231 EXOs have high expression of CD44 marker and low expression of EpCAM and HER2 markers. The EXOs are positive for all three EXO markers (CD81, CD63, and CD9).

FIG. 18A, FIG. 18B, FIG. 18C, and FIG. 18D are graphs showing flow cytometry analysis of the expression of surface markers on breast cancer SKBR3 cells with IgG control. FIG. 18A shows the distribution of the fluorescence signals and scattering signals from SKBR3 cells labeled with PE-conjugated EpCAM antibodies in terms of cell counts. FIG. 18B shows the distribution of the fluorescence signals and scattering signals from SKBR3 cells labeled with PE-conjugated CD44 antibodies in terms of cell counts. FIG. 18C shows the distribution of the fluorescence signals and scattering signals from SKBR3 cells labeled with PE-conjugated HER2 antibodies in terms of cell counts. FIG. 18D shows the distribution of the fluorescence signals and scattering signals from SKBR3 cells labeled with PE-conjugated IgG control proteins. The results show that SKBR3 cells have high expression of EpCAM and HER2 markers, and low expression of the CD44 marker.

FIG. 19 is a graph showing the size distribution of SKBR3 EXOs as measured by Nanoparticle Tracking Analysis (NTA). The size for SKBR3 EXOs was 165±38 nm (mean±standard deviation).

FIG. 20A and FIG. 20B are graphs showing the detection and protein profiling of SKBR3 EXOs using cREVA. FIG. 20A shows averaged SERS spectra (n=3) targeting different surface proteins (EpCAM, CD44, HER2, CD1, CD63, and CD9) on SKBR3 EXOs. FIG. 20B shows the expression profile of the target proteins on SKBR3 EXOs based on the intensity values of the 1497 cm⁻¹ peak in the SERS spectra shown in FIG. 20A. The results show that SKBR3 EXOs have high expression of EpCAM and HER2 markers and low expression of the CD44 marker. The EXOs are positive for all three EXO markers (CD81, CD63, and CD9).

FIG. 21A, FIG. 21B, FIG. 21C, and FIG. 21D are graphs showing flow cytometry analysis of the expression of surface markers on normal breast MCF12A cells with an IgG control. FIG. 21A shows the distribution of the fluorescence signals and scattering signals from MCF12A cells labeled with PE-conjugated EpCAM antibodies in terms of cell counts. FIG. 21B show the distribution of the fluorescence signals and scattering signals from MCF12A cells labeled with PE-conjugated CD44 antibodies in terms of cell counts. FIG. 21C show the distribution of the fluorescence signals and scattering signals from MCF12A cells labeled with PE-conjugated HER2 antibodies in terms of cell counts. FIG. 21D shows the distribution of the fluorescence signals and scattering signals from MCF12A cells labeled with PE-conjugated IgG. The results show that MCF12A cells are EpCAM positive with low expression of CD44 and HER2 markers.

FIG. 22 is a graph that shows the size distribution of MCF12A EXOs as measured by NTA. The size for MCF12A EXOs was 161±40 nm (mean±standard deviation).

FIG. 23A and FIG. 23B are graphs showing the detection and protein profiling of MCF12A EXOs using cREVA. FIG. 23A shows averaged SERS spectra (n=3) targeting different surface proteins (EpCAM, CD44, HER2, CD1, CD63, and CD9) on MCF12A EXOs. FIG. 23B shows the expression profile of the target proteins on MCF12A EXOs based on the intensity values of the 1497 cm⁻¹ peak in the SERS spectra shown in FIG. 23A. The results show that MCF12A EXOs are EpCAM positive with weak expression of CD44 and HER2 markers. The EXOs are positive for all three EXO markers (CD81, CD63, and CD9).

FIG. 24 is a graph showing a comparison of the expression profiles of surface proteins on EXOs derived from different breast cancer cell lines (MM231 and SKBR3) and normal breast cell line (MCF12A). All cell lines have positive expression of the three EXO markers CD81, CD63, and CD9 but with different levels. The results identified that CD44 as MM231 EXO cancer marker and HER2 as SKBR3 EXO cancer marker. These marker expression patterns on EXOs reflect those on their originating cells as measured by flow cytometry analyses, which suggests that EXOs are a resource of cancer markers for diagnostics.

FIG. 25A-D provide comparisons of surface marker expressions of EpCAM (A, B) and HER2 (C, D) between cancer patients and healthy donors. FIG. 25A shows average SERS spectra (n=3) from each subject for the EpCAM marker. FIG. 25B shows the protein expression profiles based on the data in FIG. 25A. The p-value between cancer patients and healthy donors for EpCAM is 7.4×10⁻¹¹. FIG. 25C shows average SERS spectra (n=3) from each subject for the HER2 marker. FIG. 25D shows the protein expression profiles based on the data in FIG. 25C. The p-value between cancer patients and healthy donors for HER2 is <2.2×10⁻¹⁶.

FIGS. 26A and 26B are a set of receiver operation characteristics (ROC) curves generated based on patient profiling data in FIG. 25.

FIG. 27 is a schematic diagram of the SERS-SVT method for detection and quantification of targeted EXO proteins. EXOs are captured directly from diluted biofluids with an EXO marker. Targeted surface proteins are recognized with primary antibodies and then SERS AuNR-secondary antibodies. EXOs will be imaged under dark field with white light illumination to detection and localize EXOs. EXOs will also be imaged under Raman mode to detect the targeted proteins on EXOs. By analysis of the dark field and SERS images, the protein level on each exosome can be quantified. By statistical analysis of multiple exosomes, the expression profile of the targeted proteins on the EXOs from specified origin can be obtained.

FIGS. 28A to 28D are photographic images and a schematic diagram of the fabrication of the chamber slide for multiple exosome analysis. FIG. 28A is an image an Au-coated glass slide. FIG. 28B is a schematic diagram of the design of a multi-well cassette. FIG. 28C is an image of a multi-well cassette fabricated by a 3D printer. FIG. 28D is an image of the chamber slide formed by the cassette and Au-coated glass slide.

FIGS. 29A to 29G are a schematic diagram of the process of capturing an exosome and multiple images of captured exosomes. FIG. 29A is a schematic diagram of exosome capture onto the Au chamber slide. FIG. 29B is a fluorescence image of exosomes derived from MM231 breast cancer cells captured with anti-CD81 antibodies. FIG. 29C is a fluorescence image of exosomes derived from SKBR3 breast cancer cells captured with anti-CD81 antibodies. FIG. 29D is a fluorescence image of exosomes captured with anti-CD81 antibodies from a plasma sample of a first breast cancer patient. FIG. 29E is a fluorescence images of exosomes with captured anti-CD81 antibodies from a plasma sample of a second breast cancer patient. FIG. 29DF is a fluorescence image of exosomes captured with anti-CD81 antibodies from a plasma sample of a third breast cancer patient. FIG. 29G shows fluorescence image of exosomes derived from the patient as shown in FIG. 29F captured with IgG control protein.

FIGS. 30A and 30B illustrate the detection of a targeted cancer marker using the presently disclosed methods. FIG. 30A is a schematic diagram showing the labeling of EXOs with SERS AuNR tags. FIG. 30B is a chart illustrating the SERS signals from SKBR3 EXOs targeting HER2 with the SERS AuNR tags.

FIGS. 31A and 31B illustrate the instrumentation for signal collection. FIG. 31A is a schematic diagram of the optical microscopic system for data collection. FIG. 31B is a photograph of the optical microscopic system for data collection.

FIGS. 32A to 32G are examples for data collection (A-C) and analyses (D-F) with SERS-SVT. EXOs are derived from MM231 cells and labeled with CD44 primary antibody and then SERS AuNR tag secondary-antibody for CD44 detection. FIG. 31A is a dark field mask image. FIG. 31B is a SERS target image. FIG. 32C is a chart of the analysis performed using the Image J software with ROI function for overlaying the mask and target images. FIG. 32D is an image showing the target image with outlined areas at the locations of exosomes in the mask image. FIG. 32E is a graph illustrating the pixel intensity of the EXOs in FIG. 32D. FIG. 32F shows the population density histogram with the IgG control.

FIGS. 33A to 33C show protein profiling of EXOs derived from SKBR3 breast cancer cells. FIG. 33A is a graph illustrating the density population profile of HER2 on the EXOs. FIG. 33B is a graph illustrating the density population profiles of CD44 on the EXOs. FIG. 33C is a graph illustrating the density population profiles of EXOs with IgG as the control for the primary antibody.

### DETAILED DESCRIPTION OF THE INVENTION

This invention features a transformative technology for the detection and quantitative surface protein profiling of extracellular vesicle (EV or EVs) (e.g. exosome (EXO or EXOs), microvesicle (MV or MVs), apoptotic body) using surface enhanced Raman scattering (SERS) nanotags.

This technology, named Raman Extracellular Vesicle Assay (REVA), features the use of highly sensitive and highly specific surface enhanced Raman scattering gold nanorod (SERS AuNR) tags to label EVs and quantitatively detect EV surface proteins with SERS spectroscopy. The assay is advantageously efficient and can be used in combination with a low cost portable EV array device that provides for the analysis of the molecular expression pattern of target-specific surface proteins present on EVs and other membrane-bound vesicles from any biological sample (e.g., any cell or tissue, including body fluids, such as blood, urine, saliva, cerebrospinal fluid), or from any biological source (e.g., a human or non-human mammal). REVA may be used to detect many types of diseases (e.g., cancer, neurodegenerative disorders, such as Alzheimer's disease, Parkinson's disease) and characterize the molecular expression patterns of proteins from any biological sample. REVA provides the first application of SERS nanotags for the analysis of EVs and membrane bound vesicles.

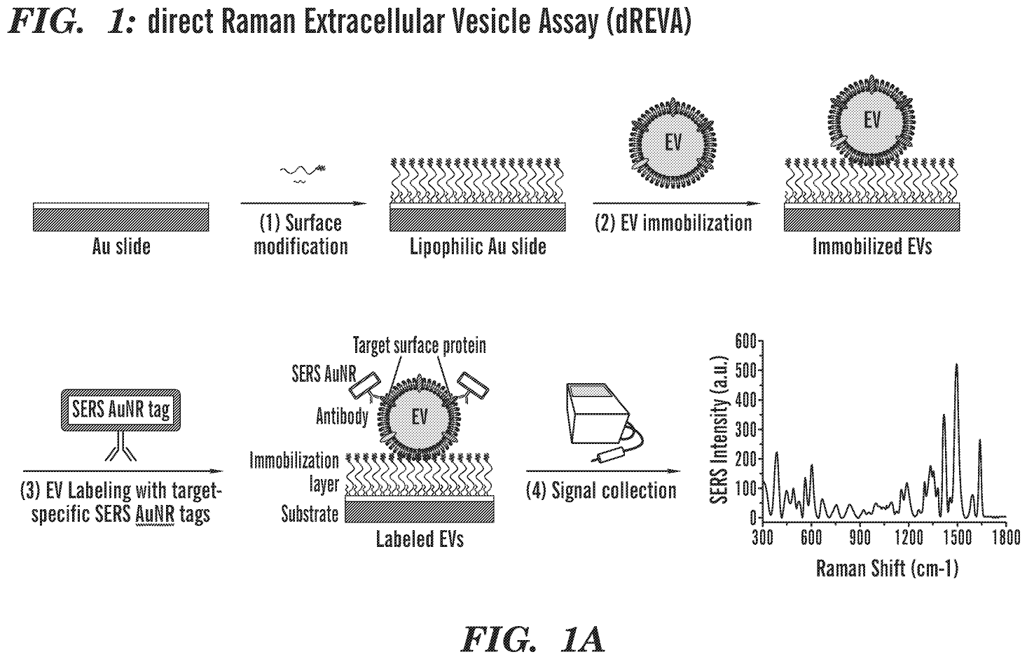

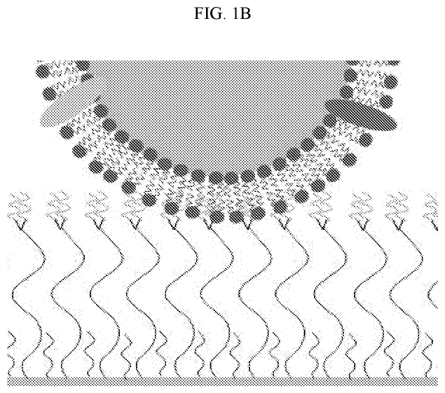

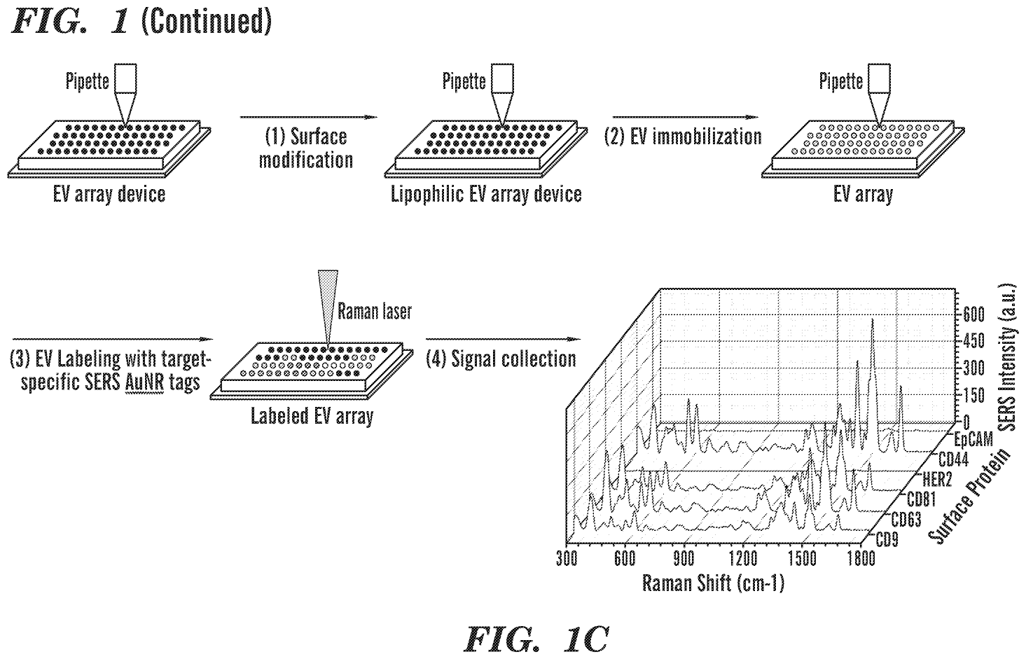

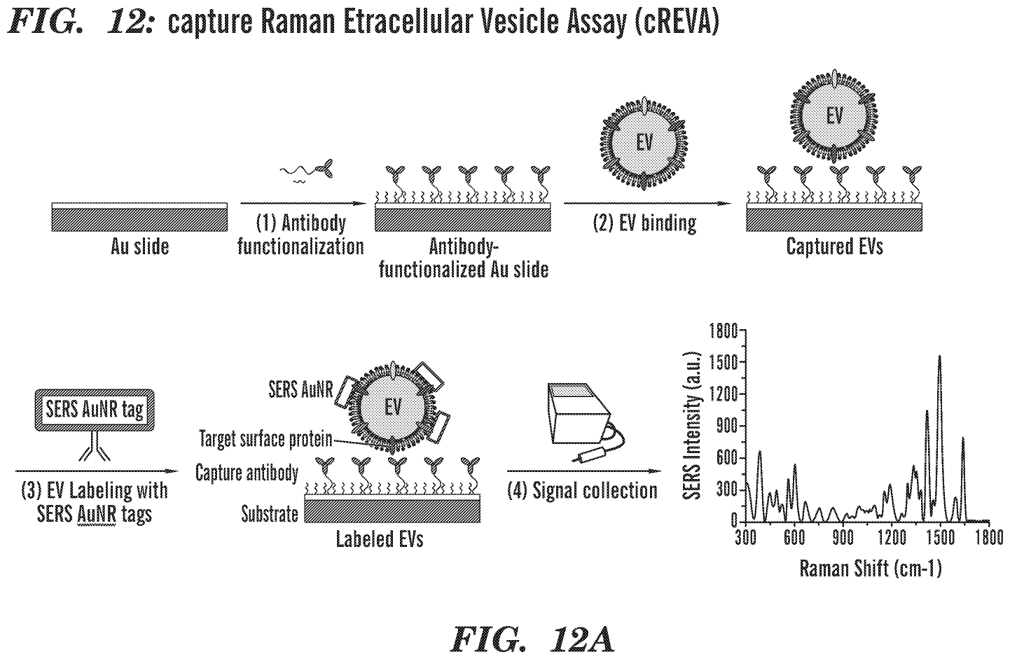

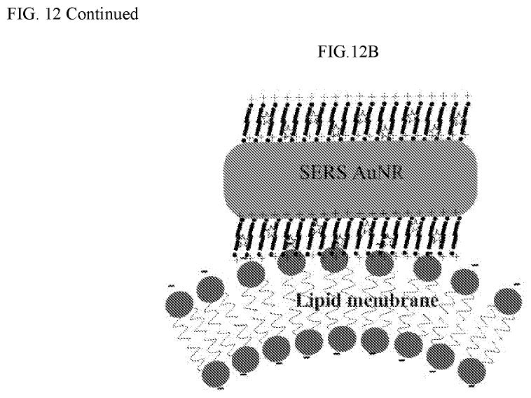

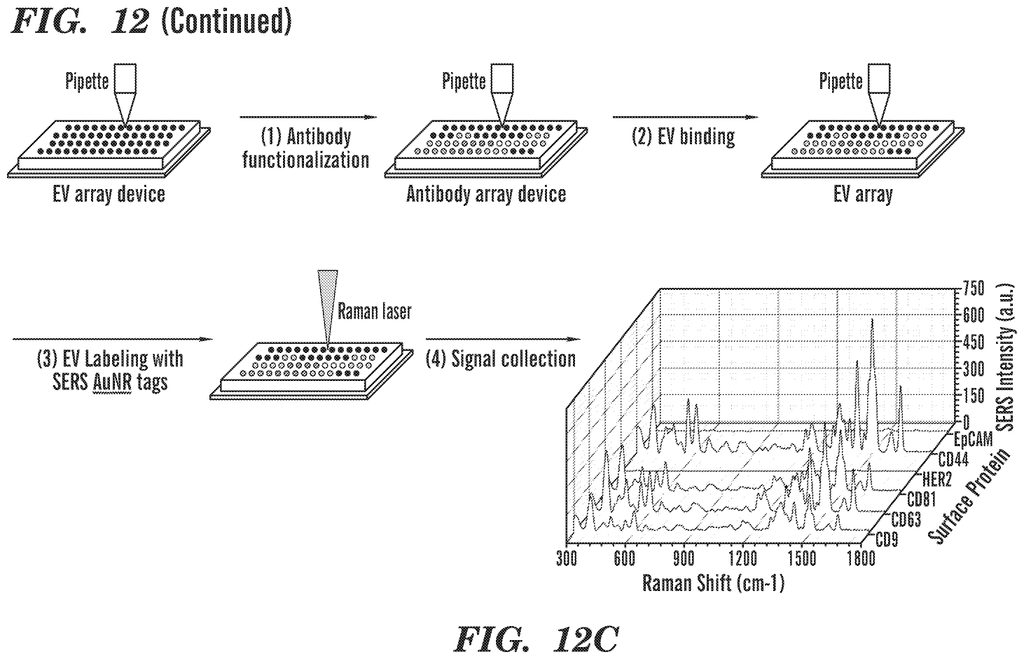

REVA involves four major components: (1) extracellular vesicles (EVs) (or any other membrane bound vesicles (MBVs), cell, bacteria, virus, or similar particle isolated from a biological sample; (2) a device that immobilizes or captures EVs in a multiplex fashion (“EV array device”); (3) a labelling agent (e.g., Raman reporter) that provides for EV detection by SERS spectroscopy; and (4) a Raman spectrometer that collect signals. Depending on how the EVs are labeled with the labeling agent, the REVA is typically performed by direct Raman Extracellular Vesicle Assay (dREVA) (FIG. 1) or by capture Raman Extracellular Vesicle Assay (cREVA) (FIG. 12).

Combining SERS detection with high sensitivity and specificity, and with an EV array device having high portability and high efficiency, allows for the innovative REVA technology to perform dozens of tests on a single palm size device from microliter sized samples with high sensitivity. For example, as described below, dREVA can detect EXOs at a concentration of 1×10⁶ EXO/mL that is over 1000 times lower than the concentration of EXOs in human plasma (≥10⁹ EXO/mL). This easy-to-operate, low cost, portable, efficient, highly sensitive, and highly specific REVA technology will facilitate molecular analysis of EVs, especially EXOs, and is useful in basic and clinical EV research, not only for marker discovery, but for providing insights into the role of EVs in disease development. It will open new avenues for developing new generation cancer liquid biopsy to diagnose cancer, monitor cancer progression, and monitor patient treatment responses in real-time. The REVA technology can be used world-wide, especially in limited-resource research and clinical environments and will advantageously impact cancer diagnostics and personalized treatment.

Another feature of the invention is the use of high throughput 3D printing technology to print a protein array to capture membrane bound vesicles in a target-specific manner on a functionalized gold chip, and label and detect membrane bound vesicles in a high throughput fashion with highly sensitive surface enhanced Raman scattering (SERs) small gold nanorods. This simple, inexpensive, and portable assay offers dozens of test sites on a single palm size chip from microliter samples within two hours, with an unprecedented limit of detection. For example, as described below, the methods have a limit of detection down to 200 exosomes.

### SERS Nanotags for EV Protein Analysis

The invention provides the first application of surface enhanced Raman scattering (SERS) nanotags for EV analysis. SERS provides for the enhancement of Raman signals of small organic molecules by roughened metallic surface via electromagnetic and chemical mechanism (K. Kneipp et al. J. Phys. Condens. Matter 2001, 14, R597). It can be used to detect EXO molecular constitutes, such as protein, carbohydrates and lipids by enhancing the Raman signals of the molecular constitutes of EXOs (L. Tirinato et al. Microelectron. Eng. 2012, 97, 337; C. Lee et al. Nanoscale 2015, 7, 9290; S. Stremersch et al. Small 2016, 12(24), 3292; J. Park et al. Anal. Chem. 2017, 89, 6695). In contrast, this invention features the use of SERS nanotags for quantitative surface protein profiling of EVs. SERS nanotags are plasmonic nanoparticles (e.g. gold and silver nanoparticles), such as gold nanoparticles coated with Raman reporters such as organic dyes. SERS nanotags provide for the highly sensitive detection of targets of interest with a known SERS spectrum of the Raman reporter (Y. Wang et al. Chem. Rev. 2013, 113(13), 1391). For example, circulating tumor cells in whole blood can be detected at a LOD of 1-2 cell/mL blood using iron oxide-gold core-shell nanoparticles carrying QSY21 reporter (S. Bhana et al. Nanomedicine(Lond) 2014, 9(5), 593). This high sensitivity is due to the strong Raman enhancement of the Raman reporter by the plasmonic nanoaprticles and the abundacy of the Raman reporters on the plasmonic nanoparticles.

Compared to current methods for surface protein analysis of EVs including surface plasmon resonance sensing (SPR technique) (H. Im et al., Nat. Biotechnol. 2014, 32(5), 490; L. Grasso et al., Anal. Bioanal. Chem. 2015, 407, 5425; A. A. I. Sina et al., Sci. Rep. 2016, 6, 30460; A. Thakur et al, Bioelectron. 2017, 94, 400) and resonance light scattering sensing (K. Liang et al. Nat. Biomed. Engineer. 2017, 1, 0021), the use of SERS nanotags for detection has at least two major advantages. First, data analysis is extremely simple. SERS provides fingerprint signals that distinguish interferences from biological background. The SERS spectrum only requires a simple baseline correction using a multi-segment polynomial fitting to subtract SERS background (broad continuum emission). This baseline correction is usually incorporated in the signal correction software and thus the as-acquired spectrum does not need further signal separation process for quantitative analysis. The peak intensity of the SERS spectrum from the Raman report is used to express the level of target protein on EVs. Second, signal collection is extremely fast (e.g., about a second) due to the high sensitivity of the SERS nanotags. For example, signals from 50 samples on a single device can be collected within about 1 minute, which is extremely fast and efficient.

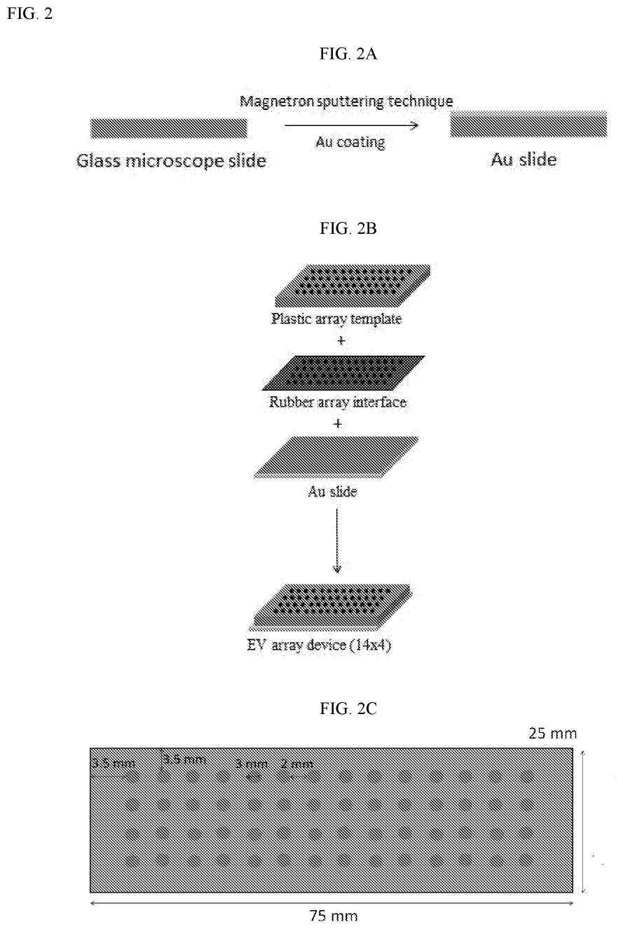

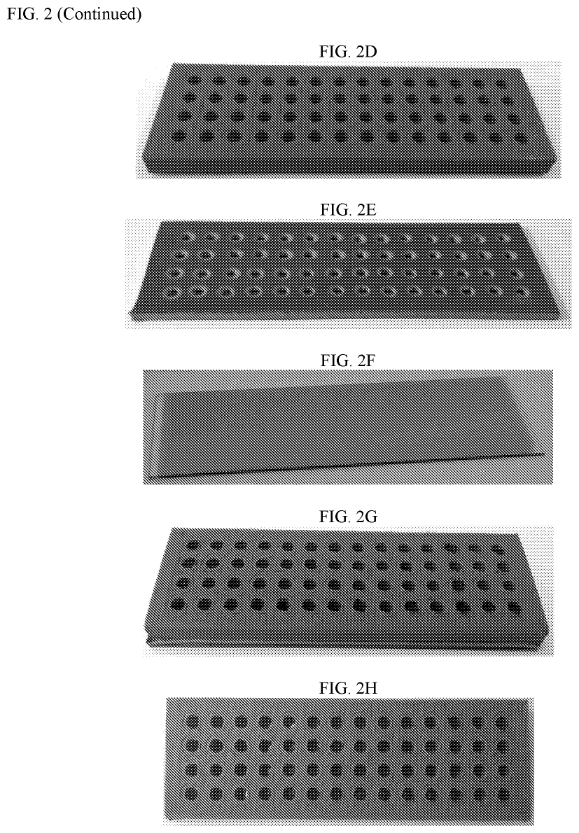

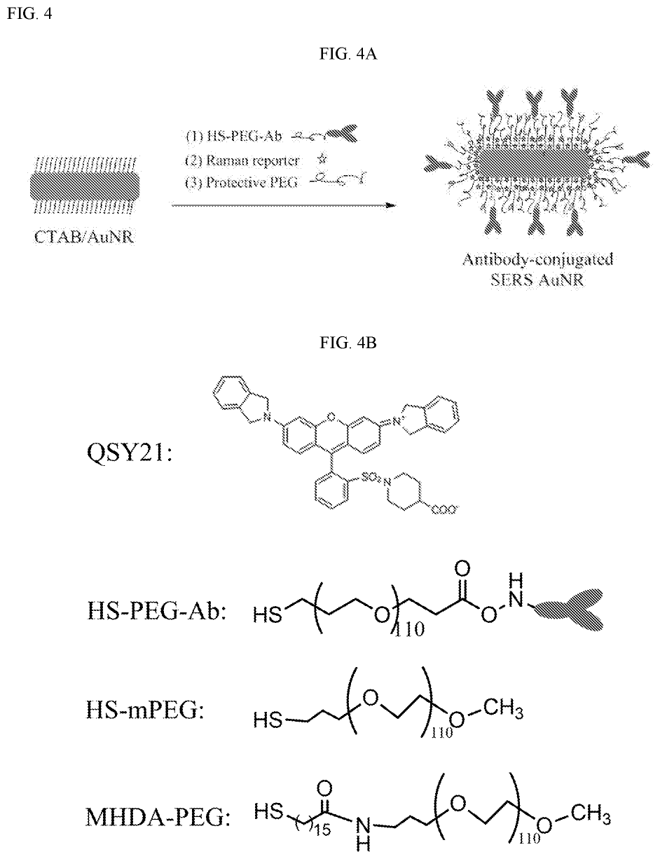

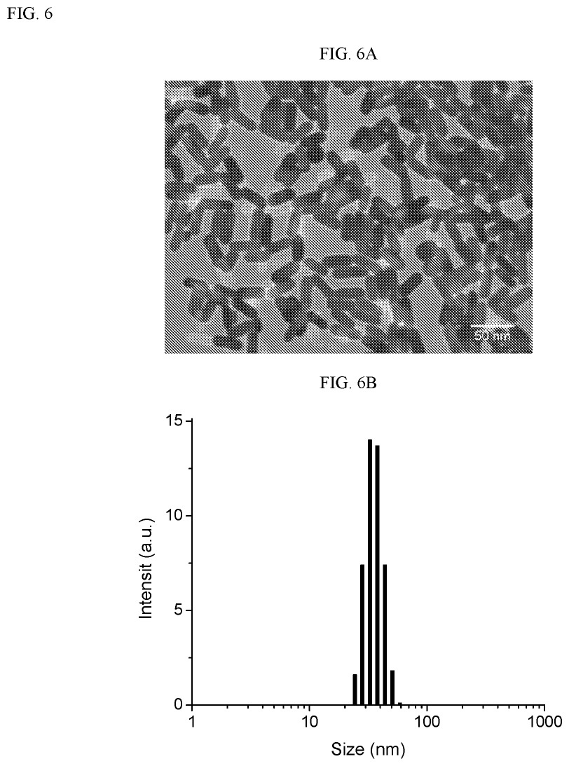

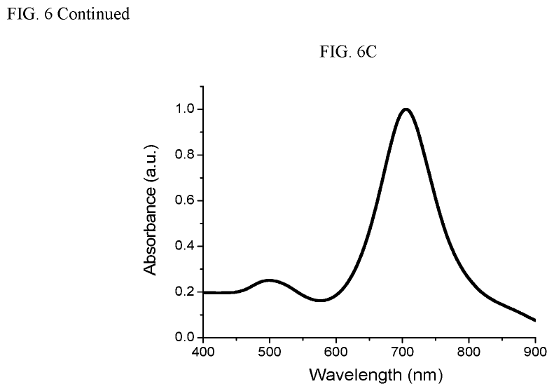

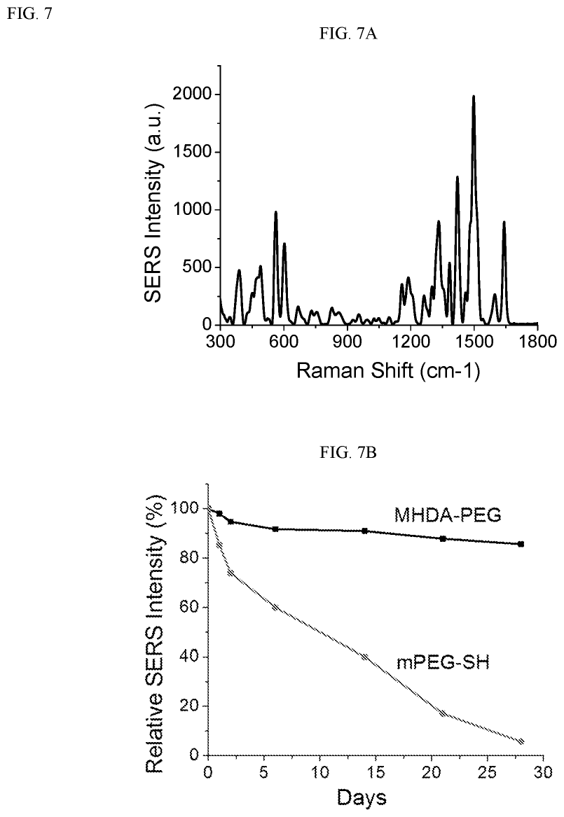

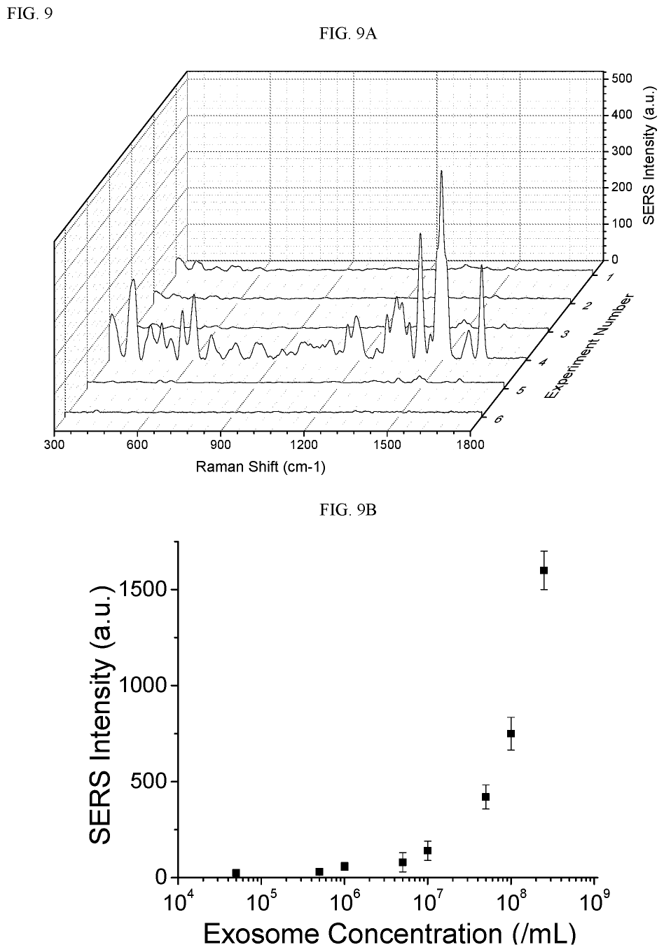

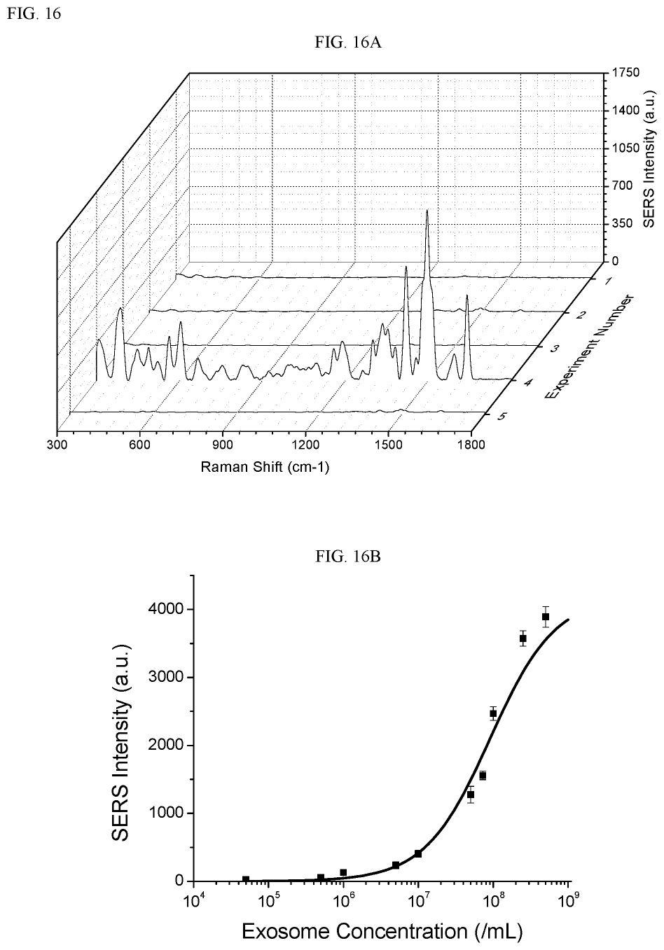

An example of the plasmonic nanoparticles is anisotropic small gold nanorods (FIG. 6). The rod-shaped nanoparticles feature high SERS activity by concentrating high electromagnetic fields at the ends of the rods (G. Hao et al. J. Chem. Phys, 2004. 120(1): p. 357-366). The AuNRs were synthesized using the classic seed-mediated growth method (X. Huang et al. J. Am. Chem. Soc. 2006, 128(6), 2115). The AuNRs have an average dimension of 35 nm in length and 12 nm in width, with a localized surface plasmon resonance (LSPR) at 720 nm. An example of a Raman reporter is the organic dye QSY21 (FIG. 4). QSY21 is non-fluorescent and have strong and fingerprinting SERS spectrum (FIG. 7A) that provides high specificity and high sensitivity detection of target proteins of interests. The QSY21-coated SERS AuNRs offers highly sensitive detection of EVs, with limit of detection (LOD) for EXO detection at 1×10⁶ EXOs/mL in the dREVA (FIG. 9B) and 2×10⁶ EXOs/mL in the cREVA (FIG. 16B). The detectable level of concentration of EXOs using the SERS AuNRs is over 1000 times lower than the typical concentration of EXOs in human plasma.

### Array Device for Multiple Analyses

Another feature of the invention is an EV device that allows for simultaneous processing and detection of multiple samples. An example of this EV device was fabricated with an Au slide and a template array (FIG. 2). The Au slide can be fabricated using a magnetron sputtering technique by depositing a thin film of Au atoms onto a standard glass microscope slide that is 75×25×1 mm (length×width×thickness). An Au layer can be used to facilitate chemical surface modification for EV immobilization or capture. The thickness of the Au layer can vary, but a typical thickness of 10 nm has been used. This 10-nm thick Au film is optically transparent and thus can be used for optical imaging as well. The Au slide can be divided into multiple measurement sites with the use of a plastic array template. The plastic template, which is made of polylactic acid can be fabricated with 3D printers. The wells in the template can be varied based on users need. The maximal throughput for manual sample process is 14×4 wells, which allows for analyzing 56 samples at the same time. The well size is 3 mm in diameter and inter-well distance is 2 mm. The template is assembled onto the Au slide to form EV array divide with a rubber array interface that helps fix the plastic template array onto the Au slide.

### Lipophilic EV Immobilization

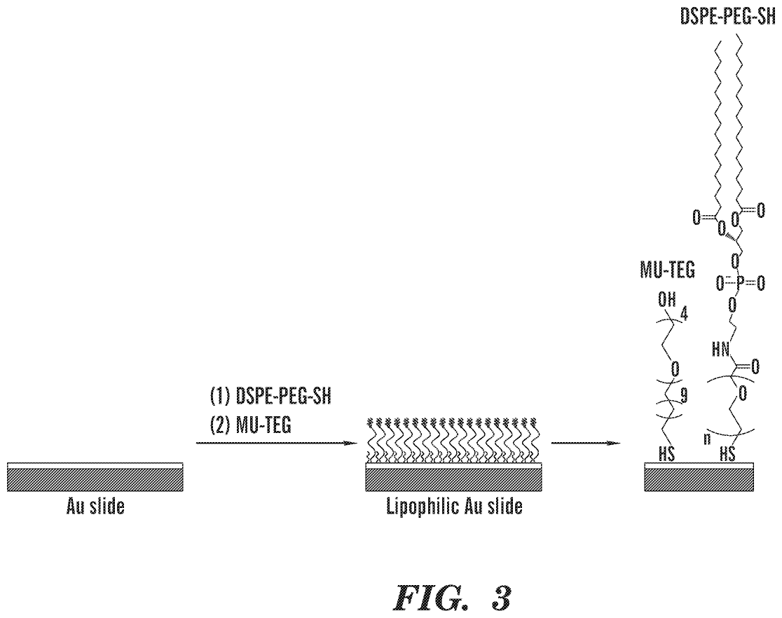

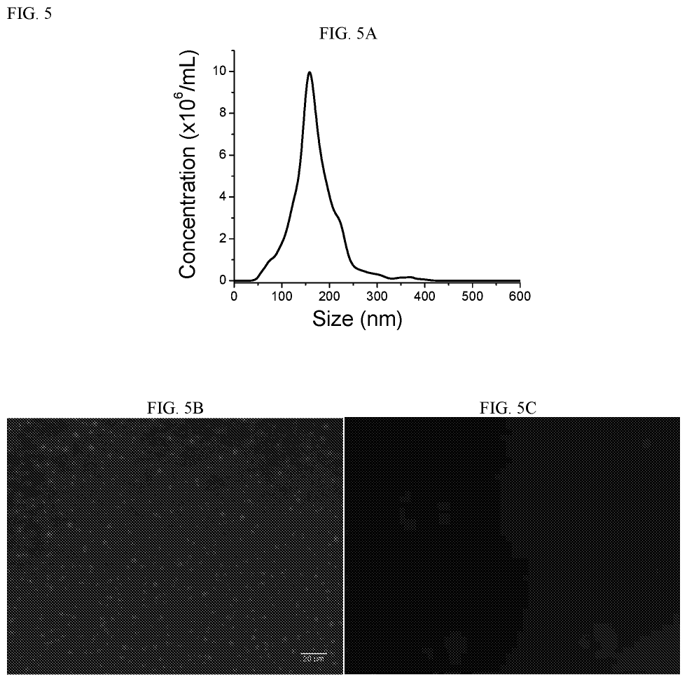

In dREVA, EVs are immobilized on an array using a lipophilic chemical layer on the device and then labeled and detected using SERS nanotags. Lipophilic molecules with an alkyl chain have high affinity for the lipid bilayer of molecules (e.g., EVs, cells, organelles, membranes) through hydrophobic interactions between the lipid membrane of the target and the lipophilic molecules on the substrate. This invention features lipophilic molecule 1,2-distearoyl-sn-glycero-3-phosphoethanolamine-conjugated polyethylene glycol thiol (DSPE-PEG-SH, MW 5000) combined with a hydrophilic short chain of 11-mercaptoundecyl tetra(ethylene glycol) (MU-TEG) (FIG. 3). While the thiol groups from the DSPE-PEG-SH bind to the Au surface, the DSPE portions interact with the EV lipid membrane for binding to EVs. The MU-TEG molecules can be used to saturate the Au surface to eliminate nonspecific interactions. This unique combination of DSPE-PEG-SH and MU-TEG offers highly specific capture (FIG. 5) and SERS detection (FIG. 9A) of EVs. This surface modification can immobilize EXOs with over 87% efficiency by incubation of an EV solution on the device for only 30 minutes.

### Target-Specific EV Capture

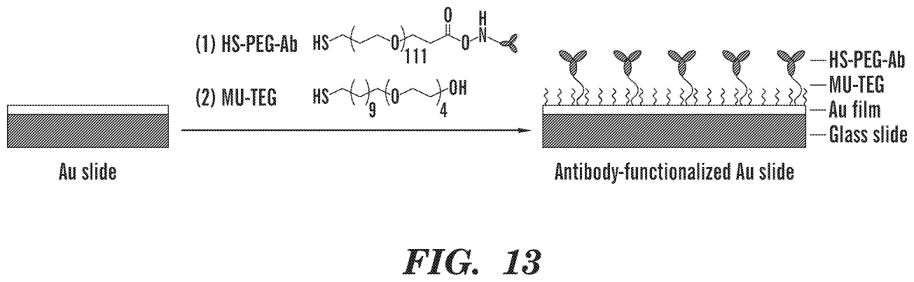

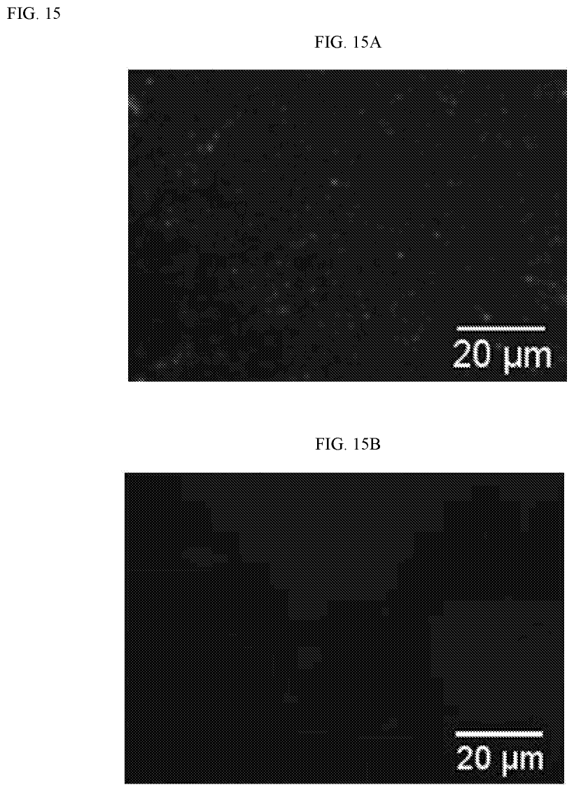

In cREVA, EVs are captured on the array device by fixing target specific capture molecules (e.g., ligands), such as antibodies, on the Au surface of the device. For example, the antibodies can be conjugated to a PEG-SH linker in advance via an amide bond by linking commercially available HS-PEG-NHS MW5000 (e.g. Nanocs Inc.) with antibodies. The HS-PEG-Ab binds to the Au slide surface via Au—S bond, leaving external antibodies for specific recognition of the surface proteins on EVs. After functionalization with HS-PEG-Ab, the Au slide is then saturated with MU-TEG to minimize nonspecific interactions (FIG. 13). EVs are captured on the functionalized device by specific binding between the antibodies on the Au slide with the target surface proteins on EVs. The combination of HS-PEG-Ab and MU-TEG offers high specific capture (FIG. 15) and SERS detection of EVs (FIG. 16A).

Labelling EVs with SERS AuNR Tags

In dREVA, the immobilized EVs via lipophilic capture were labeled with target-specific SERS AuNRs with QSY21 dye as the reporter (FIG. 1). This invention features a unique preparation method for the target-specific SERS AuNRs. The target-specific SERS AuNRs were developed by sequential binding of HS-PEG-Ab (100× molar ratio, 5 h, RT), QSY21 (10,1000× molar ratio, 15 min, RT), and 16-mercaptohexadecanoic acid-linked PEG-SH (MHDA-PEG) (100,000× molar ratio, 1 h, RT) (FIG. 4). This optimized formulation offers high stability. The SERS signal intensity (at the 1497 cm⁻¹ representative peak) decreased by 14% at 4 weeks after preparation in contrast to 93% for those stabilized with conventional methoxy-PEG-thiol (mPEG-SH 5000) (FIG. 7). This exceptional stability is due to the formation of a hydrophobic pocket by MHDA that packs QSY21 on the surface of AuNR.

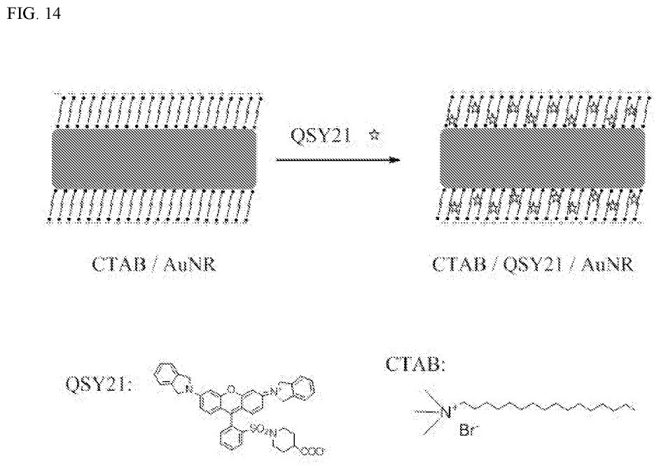

In cREVA, the ligand-captured EVs are labeled with SERS AuNR tags via electrostatic interactions of SERS AuNRs and the lipid membrane of EVs (FIG. 12). The SERS AuNRs are positively charged due to the positively charged CTAB capping materials. The lipid membrane of vesicles is negatively charged. Thus, the positively charged SERS AuNRs can bind to vesicles via electrostatic interactions. The SERS AuNRs were prepared by incubating QSY21 carboxylic acid with AuNRs (5000× molar ratio) for 15 min at RT with constant stirring (FIG. 14). Free QSY21 carboxylic acid was removed by centrifugation. QSY21 were attached to AuNRs by hydrophobic interactions with the hydrophobic pocket formed by CTAB bilayer on AuNRs. The SERS AuNR tags are aged for 2 h before use.

Signal Collection with a High-Performance Raman System

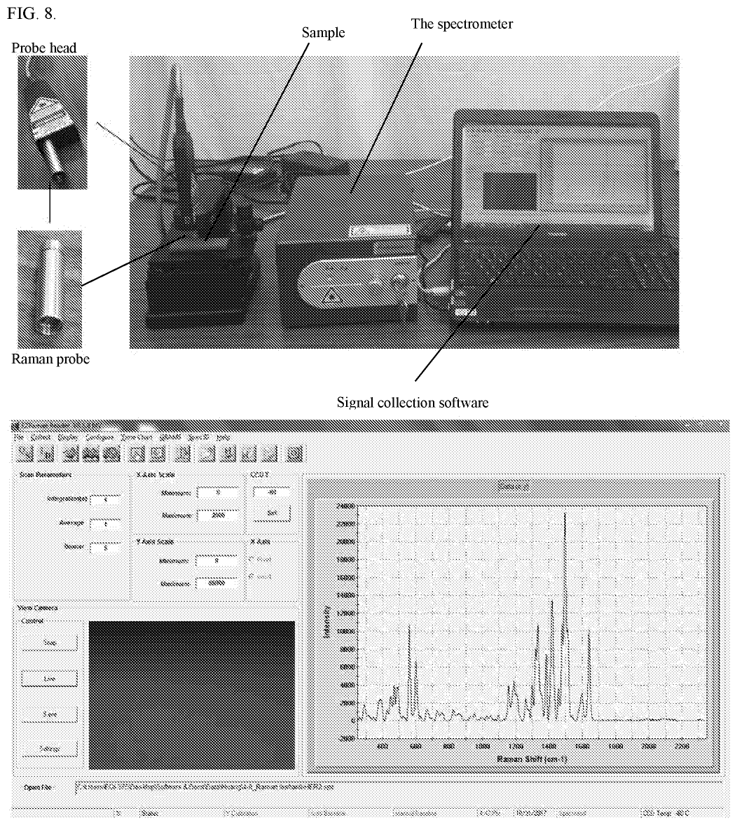

In some embodiments, a Raman spectrometer can be used for signal collection from the SERS nanotags attached on EVs. Any Raman spectrometer or Raman microscope can be used for signal collection. In some embodiments, a Raman spectrometer is portable, low cost and high throughput. An example of such Raman system is ProRaman-L high performance spectrometer from TSI (FIG. 8). The spectrometer features a high sensitivity CCD spectrograph with CCD cooling to −60° C., HRP-8 high throughput fiber-optical Raman probe from a 785 nm diode laser with O.D.>8 Rayleigh rejection, and high signal to noise characteristics. The Raman probe is portable and can offer different laser spot size at focus from 60 to 300 μm depending on the lens. The REVA uses a 200 μm as it is the most sensitive one when detecting EXOs in EV array device. The power is adjustable from 0 to 250 mW. Integration time per spectrum is as fast as 50 ms. The system provides automatic background (baseline) correction using a multi-segment polynomial fitting by clicking “background subtraction” under “Configure” in the EZRaman Reader V8.1.8 MV signal acquisition software. Thus, the as-acquired spectrum is background subtracted (subtract broad continuum emission background) and is ready for use without further signal processing.

### Single Vesicle Detection

In some aspects of the present disclosure, methods for detecting single vesicles are provided that use single vesicle technology (SVT), which is based on surface enhanced Raman scattering (SERS) imaging to probe tumor-derived exosomes in the presence of non-tumor exosomes. This approach is referred to as SERS-SVT. In some embodiments, small SERS gold nanorod (AuNR) tags are used to label targeted surface protein markers on exosomes that will be captured directly from body fluids. Dark field imaging is used to localize the captured exosomes in a multi-well chamber slide and SERS imaging is used to detect the proteins on single exosomes. By analyzing the dark field mask image and the SERS target image, the expression profile of targeted proteins may be obtained that informs the amount and the protein level of the exosome subpopulation positive to the targeted protein. SVT is much more sensitive and provide valuable information that is not available in current bulk methods. SVT can identify cancer-derived EXOs that are undetectable by current bulk methods, thereby detecting cancer early. SVT can quantify the fraction of tumor-derived EXOs, which is critical in monitoring tumor progression. Further, SVT can reveal EXO subpopulations and discern compositional heterogeneity, which are very useful to understand tumor heterogeneity and help personalized treatment.

Tetraspanin CD81 is an EXO marker that differentiates EXOs from other types of extracellular vesicles; therefore, CD81 antibody can be used to capture EXOs from a biofluid. Other markers can be used to isolate EXOs including, but not limited to, ALIX, TSG101, and other tetraspanins such as CD63 and CD9. The method can directly capture EXOs with, for example, monoclonal antibodies from plasma and other biofluids without EXO pre-purification. In some embodiments, the antibody is conjugated to a polyethylene glycol thiol (PEG-SH) linker (MW=5000) by reacting HS-PEG-NHS with antibody. In some embodiments, the antibody conjugated to the linker may be purified by filtration centrifugation.

In some embodiments, capture of EXOs comprises immersing a chamber slide having an Au surface in composition comprising an antibody that specifically binds an antigen associated with an exosome, wherein the antibody is linked with PEG-SH. In some embodiments, this step is followed by a wash step with PBS. The chamber slide may then be immersed in a composition comprising an agent that inhibits or reduces nonspecific binding to the slide. In some embodiments, the agent is 11-mercaptoundecyl tetra (ethylene glycol) (MU-TEG). To capture the EXOs in a sample, the sample is incubated on the chip for a sufficient period of time to capture the EXOs in the sample. In some embodiments, the incubation period is about 2 hours. After immobilization, EXOs can be visualized with membrane staining agent such as DiO and DIB.

### Diagnostics

In some embodiments, the profiling of MBVs and/or EVs may be used as a diagnostic tool. Subjects having or at risk of developing a disease are diagnosed using any method known in the art. In particular embodiments, a subject is identified as being at risk to develop the disease. For example, the molecular profiling of labelled MBVs and/or EVs on a Raman spectrometer of a sample may be used to determine a subject who is at risk of acquiring a disease by comparing the subject's molecular profile to a different subject who has already been determined to not be at risk of acquiring the disease. In other embodiments, a subject is identified as having a disease.

For example, the molecular profiling of labelled MBVs and/or EVs on a Raman spectrometer of a sample may be used to determine a subject who has a disease by comparing the subject's molecular profile to a different subject who has already been determined to have the disease.

### Kits

The invention provides kits that include a device (e.g., a microcopy slide, a chip, an Au-array device, or a bead) and an agent (e.g., a long chain lipophilic polymer and a short chain hydrophilic molecule). In some embodiments, the device contains a gold-coated glass microscope slide, an array template, and a rubber array interface.

In some embodiments, the kit comprises a sterile container which contains AuNRs, a Raman reporter, a nanotag stabilizer, and one or more target-specific functionalized antibodies. Such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### Example 1: Direct Raman Extracellular Vesicle Assay (dREVA)

Schematic illustrations of the methodology of the direct Raman Extracellular Assay (dREVA) is shown in FIG. 1A, FIG. 1B, and FIG. 1C. FIG. 1A and FIG. 1B show the principle of the assay and FIG. 1C shows the procedures when using the assay. The assay contains four sequential steps: (1) Lipophilic surface modification of Au slide; (2) EV immobilization; (3) EV labeling with the target-specific SERS AuNR tags; and (4) Signal collection with a Raman spectrometer. EVs are immobilized on the surface of Au slide via hydrophobic interactions between the lipid membrane of EVs and the hydrophobic segments of the lipophilic molecules grafted on the surface of Au slide. The EV device has multiple wells that allow for analysis of different proteins or different EVs on the same device simultaneously. The array takes about 2 about 3 h. The method gives a quantitative measurement of the target surface proteins of interests on EVs and thus a quantitative surface protein expression profile of EVs. The results (i.e. the protein expression profile on EVs) can be used to understand EV biology, diagnose disease (e.g. cancer), monitor disease progression, and monitor patient treatment response.

The Au slide is fabricated by depositing 10-nm thick Au film onto a standard glass microscope slide with a magnetron sputtering technique (FIG. 2A, FIG. 2B, FIG. 2C, FIG. 2D, FIG. 2E, FIG. 2F, FIG. 2G, and FIG. 2H). The Au film was designed to be 10 nm in thickness because an Au film at such thickness is optically transparent. Thus, the Au slide can be used for SERS-based detection, and for optical imaging such as fluorescence imaging of EVs captured on the slide. The microscope glass slide had dimensions of 75×25×1 mm (L×W×H). The EV array device was developed by assembly of the Au slide, a plastic template array, and a rubber array interface (FIG. 2B, FIG. 2C, FIG. 2D, FIG. 2E, FIG. 2F, FIG. 2G, and FIG. 2H). In the experiments of this example, a low cost 3D-printing technology was used to make an array template to divide the Au slide into multiple wells to analyze multiple samples simultanously. A 3D printer is as cheap as few hundred dollars and thus availabe to a wide range of populations. For example, the Lulzbot mini desktop 3D printer is $500.00. In the experiments of this example, a MakerBot Replicator PC 3D printer was used that costs $2,500.00. The Lulzbot mini desktop and MakerBot Replicator PC 3D printers have no difference on performance. The printer can print wells with sizes as small as 50 μm. To facilitate manual operation with the regular pipette, the size of the well was optimized to be 3 mm in diameter. An optimal inter-well distance was determined to be 2 mm. To avoid sample contamination during handling, the distance of the array to the edge of the template was set to 3.5 mm. Based on this design, the experiments of this example fabricated a standard 14×4 array that provided 56 measurement sites per device. The template material was polylactic acid. The dimensions of the rubber array interface were 75×25×1.6 mm (L×W×H) with 56 wells (3 mm in diameter). It was made to help attachment of the plastic array templae onto the Au slide. The plastic template was attached with a rubber array via a layer of glue composed of 60% silicone and 40% mineral spirit. This rubber array was made from 1.6 mm thick rubber sheet in the same dimensions as the template via punctuation. The plastic and rubber array assembly can be removed from the Au slide after use, and thus used repeatedly. The cost of each Au slide was about $25.00. The plastic and rubber array assembly was about $1.00-$2.00.

To immobilize EVs, the surface of the Au slide was grafted sequentially with long chain commercially available (e.g. Nanocs) DSPE-PEG-SH MW5000 and commercially available (e.g. Sigma Aldrich) short chain MU-TEG (FIG. 3). While the thiol groups from the DSPE-PEG-SH bound to the Au surface, the DSPE segments interacted with the EV lipid membrane for binding to EVs. The MU-TEG molecules were used to saturate the Au surface to eliminate nonspecific interactions. In the experiments of this example, systematic studies were conducted to investigate the composition and binding time of each chemical to the effects of EV immobilization with EXOs derived from breast cancer MDA-MB-231 (MM231) cells. The optimized surface modification was incubation with 1 mM DSPE-PEG-SH at RT for 1 h followed by incubation with 0.1 mM MU-TEG at RT for 30 min. Using EXOs derived from breast cancer MDA-MB-231 (MM231) cells, it was determined that 87% of EVs can be immobilized within 30 min of incubation. This surface modification is unique and has not been reported previously.

To label EVs, the experiments of this example developed and used unique antibody-conjugated SERS AuNRs using QSY21 as the Raman reporter (FIG. 4A, FIG. 4B, and FIG. 4C). Small AuNRs were synthesized using a seed-mediated growth method (X. Huang et al. 2016, 128(6), 2115) with modifications. In stead of traditional 2 h growth time, AuNRs were purified at 10 min after addition of the Au seed solution. Using a shorter growth time, AuNRs were obtained with small size (about 35 nm in length and about 12 nm in width) to meet the small EXOs. The target-specific SERS AuNR tags were formed by sequential bindings of HS-PEG-Ab, QSY21, and MHDA-PEG. In the experiments of this example, systematic studies were conducted to investigate the composition and binding time of each component to the sensitivity, specificity and stability of the formulation. The optimized procedure was: (1) binding of HS-PEG-Ab (100× molar ratio, 5 h, RT), (2) adsorption of QSY21 (10,000× molar ratio, 15 min, RT), and (3) binding of MHDA-PEG (100,000× molar ratio, 1 h, RT). The working concentration of AuNRs was 1 nM. After preparation, the solution was centrifuged at 14,000 rpm for 10 min to purify the antibody-conjugated SERS AuNR tags. The tags were suspended in PBS for use. A MHDA-PEG was used rather than conventional mPEG-SH to improve the stability of the tags. MHDA-PEG forms a hydrophobic pocket that can stabilize organic dyes in the pocket (S. Bhana et al. J. Colloid. Interface Sci. 2016, 469, 8). The HS-PEG-Ab was prepared in advance by reacting antibodies overnight with HS-PEG-NHS 5000 (1:100) at 4° C. The free HS-PEG-NHS was separated by membrane filtration with a 10 KD Nanosep filter (PALL Life Sciences). The QSY21 was the hydrolyzed form of QSY21 carboxylic acid-succinimidyl ester in water.

The labeled EVs were detected with a TSI ProRaman-L high performance spectrometer with a 785 nm laser. The Raman probe was 200 μm in diameter which covers many EVs in the well of the device. The laser beam was focused in the center of each well to collect signals of each sample. Typical signal collection parameters include integration time of 1 s and laser power of 50 mW. Baseline correction should be enabled in the signal collection software EZRaman Reader V8.1.8 MV. The signal intensity of the strongest peak at 1497 cm⁻¹ of the SERS spectrum, 11497, is used for analysis.

To account for the variations from instrumentation response and batch-to-batch nanotags, the spectrum of each nanotag solution (0.1 nM) needs to be collected before use and the 11497 value needs to be normalized to 2000 a.u., the typical value of a 0.1 nM nanotag solution. This gives a correction factor for each nanotag to correct 11497 of each sample labeled with that nanotag. The corrected values represent the level of targeted protein on EVs.

### Example 2: Efficient Vesicle-Specific Capture of EXOs with dREVA

The specificity of dREVA to immobilize membrane-bound vesicles was examined by comprising EXO immobilization between surface modification of DSPE-PEG-SH/MU-TEG and MU-PEG only. EXOs were derived from MM231 cells. FIG. 5A shows the size distribution of EXOs derived from breast cancer MM231 cells as measured by nanoparticle tracking analysis (NTA). They were isolated from conditioned cell culture medium (medium+10% EXO-free fetal bovine serum) by the gold standard isolation method—differential centrifugation (B. J. Tauro, et al. Methods 2012, 56, 293). Nanoparticle tracking analysis (NTA) shows that the EXOs were 168±49 nm (mean±standard deviation). Using 3,3′ Dioctadecyloxacrbocyanine perchlorate (DiO) membrane staining agent to image EXOs, it was found that EXOs were only immobilized when the Au slide was modified with both DSPE-PEG-SH and MU-TEG (FIG. 5B). No EXOs were found on the Au slide that was modified with MU-TEG only (FIG. 5C). It was determined that the immobilization efficiency of the DSPE-PEG-SH/MU-TEG modification is 87% when EXOs were incubated at RT for 30 min (1×10⁷/mL EXO working concentration). Thus, the experiments of this example demonstrate that EXOs can be immobilized quickly on the lipophilic Au slide with high efficiency based on specific interactions of the Au side with the lipid membrane of EVs.

### Example 3: Stable Antibody-Conjugated SERS AuNRs

Typically, AuNRs were synthesized in two steps: formation of small Au seed and growth of Au seed in an Au growth solution for 2 h to obtain AuNRs (X. Huang et al. 2016, 128(6), 2115). The AuNRs of this example were synthesized using the traditional seed-mediated growth method, but the growth time was controlled to 10 min. At this early stage of growth time, the size of AuNRs were small. The small size of AuNRs was neccessage to efficiently label the small size of EXOs. FIG. 6A shows that the AuNRs were about 35 nm in length and about 12 nm in width. Dynamic light scatterign (DLS) measurement showed a hydrodynamic diameter of 38 nm (FIG. 6B). The AuNRs have LSPR wavelength at 720 nm (FIG. 6C).

Using the AuNRs, a QSY21 reporter, CD63 antibodies and a MHDA-PEG stabilizer, the target-specific SERS AuNR tags were synthesized based on the procedure described in Example 1 (FIG. 4). A typical SERS spectrum of a 0.1 nM tags is shown in FIG. 7A (integration time: 1 s. Power: 50 mV). The spectrum shows characteristic fingerprinting of SERS signals from QSY21 reporter (S. Bhana et al. Nanoscale 2012, 4, 4939). The signal intensity of the strongest peak at 1497 cm-1 was monitored with time and compared those when the tags are stabilized with traditional mPEG-SH (FIG. 7B). The SERS signal intensity decreased by 9% at 1 week, 10% at 2 weeks, and 14% at 4 weeks after preparation for MHDA-PEG stabilized tags. In contrast, the SERS signal intensity decreased by 42% at 1 week, 60% at 2 weeks, and 93% at 4 weeks after preparation for mPEG-SH stabilized tags. Thus, the experiments of this example demonstrate that the stability of the target-specific SERS AuNRs has been dramatically improved using the new stabilization agent, MHDA-PEG.

### Example 4: Highly Specific and Sensitive Detection of EXOs with dREVA

In the experiments of this example, the specificity and sensitivity of dREVA for EV detection was examined using MM231 EXO as the model EV and CD63 as the EXO marker. FIG. 9A shows the results on the specificity test. In the specificity studies, the experiments of this example tested different surface modification of the device. The experiment numbers listed on the right side of FIG. 9A represent experiments in which: (1) the Au slide was modified with MU-TEG and incubated with SERS AuNR-CD63 antibody; (2) the Au slide was modified with MU-TEG, incubated with MM231 EXOs, and incubated with SERS AuNR-CD63 antibody; (3) the Au slide was modified with MU-TEG and DSPE-PEG-SH, incubated with SERS AuNR-CD63 antibody; (4) the Au slide was modified with MU-TEG and DSPE-PEG-SH, incubated with MM231 EXOs, and incubated with SERS AuNR-CD63 antibody; (5) the Au slide was modified with MU-TEG and DSPE-PEG-SH, incubated with MM231 EXOs, and incubated with SERS AuNR-IgG protein; and (6) the Au slide was modified with MU-TEG and DSPE-PEG-SH, and incubated with MM231 EXOs. While experiment (4) showed strong signals, other treatments showed negligible signals. These results of the experiments of this example show that the dREVA has high specificity. Nonspecific interferences from the device and the tags are negligible.

FIG. 9B shows the results on the sensitivity test via titration studies with a series of EXO dilutions. The results were presented as the dose-response curve using 11497 values at different EXO concentrations. As EXO concentration increased, the SERS signal intensity increased. Rapid signal increase was found above 1×10⁷/mL EXO concentration. Based on this titration studies, the LOD was determined to be 1×10⁶ EXOs/mL. Typically, EXO concentration in human plasma is 10⁹/mL or above (H. Shao et al. Nat. Commun. 2015, 6, 6999). Thus, the dREVA can detect EXOs at a concentration over 1000 times lower than that in human plasma. The typical working concentration is 1×10⁷ to 1×10⁸ EXO/mL and the typical working volume is 25 μL. Based on the amount of EXOs added, the immobilization efficiency, the sizes of the well and the laser spot, it was calculated that the signals were collected from 105 EXOs on the surface of Au slide in the well. This suggests that only about 100 EXOs are needs under the laser beam for detection, which is unprecedentedly sensitive compared to existing detection methods.

### Example 5: Strong Correlation of dREVA with ELISA for EXO Protein Profiling

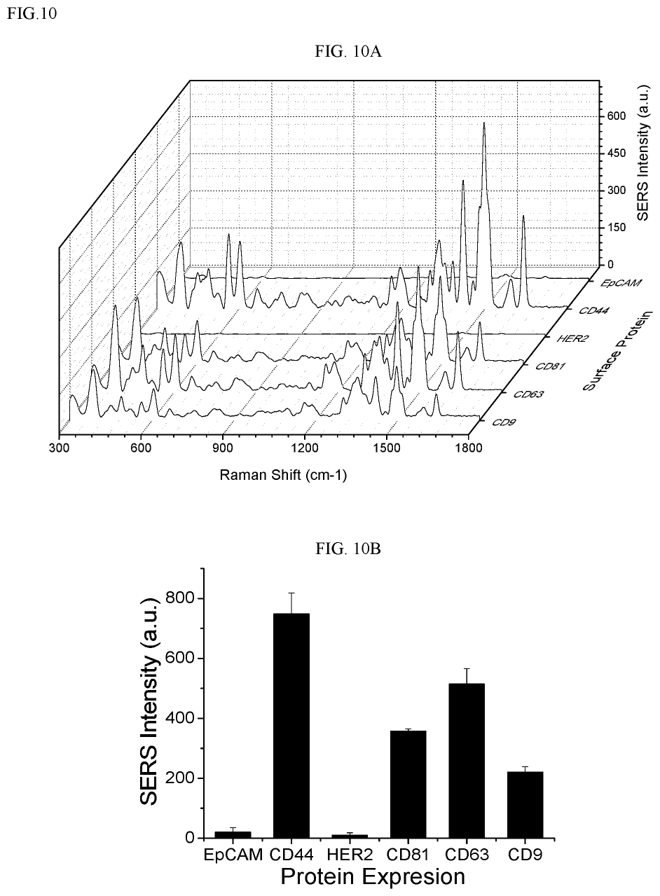

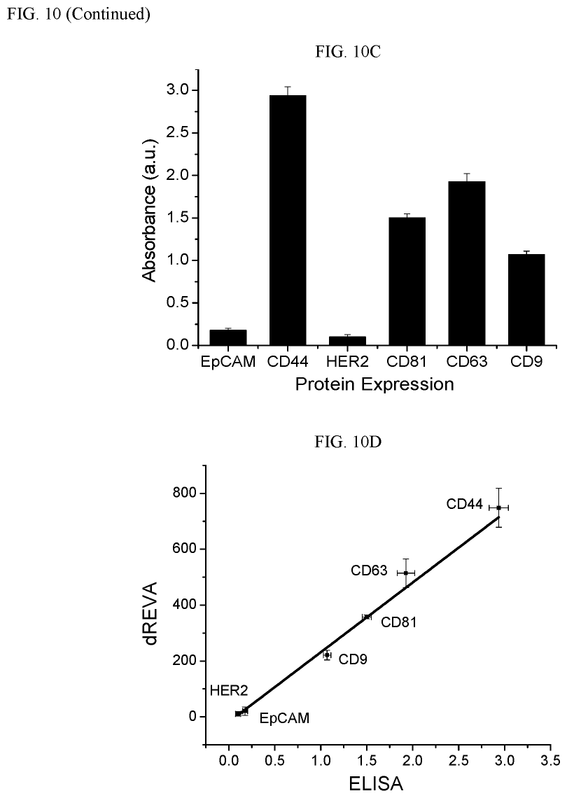

The ability of dREVA for EV protein profiling was tested and validated with traditional ELISA using MM231 EXO model. In the experiments of this example, six surface proteins were analyzed including one epithelial marker (EpCAM), two breast cancer markers (CD44 and HER2) and three EXO markers (CD81, CD63, and CD9). FIG. 10A and FIG. 10B show the averaged SERS spectrum for each target protein (n=3) and the expression profile of all six proteins on MM231 EXOs using the mean vale±SD from FIG. 10A respectively. The results show that MM231 EXOs have high expression of CD44 and the three exosome markers CD81, CD63 and CD9. They have very low expression of EpCAM and the other breast cancer marker HER2.

The dREVA was validated using the gold standard ELISA. ELISA was carried using the indirect approach, in which exosomes were adsorbed onto 96 well plates and then labeled with antibodies targeting each protein. The antibodies were recognized with HRP-conjugated secondary IgG antibody and then detected with the chromogenic substrate TMB. FIG. 10C shows protein profile on MM231 exosomes using ELISA. Similar to the results with dREVA, the EXOs have high expressions on CD44, CD81, CD63, and CD9 and low expressions on EpCAM and HER2. A quantitative comparison shows that our Raman assay has high correlation to ELISA, with correlation coefficient R² of 0.99 (FIG. 10D).

Compared to the traditional ELISA, the dREVA is much faster. The assay takes 2 about 3 h compared to >24 h for ELISA. It is also simpler by combining labeling and signal amplification into a single agent (i.e. the antibody-conjugated SERS nanotag). It is more sensitive, >10 times sensitive than ELISA. In addition, the dREVA provides point-of-care capability because of the portable nature of the Au chip and Raman spectrometer.

### Example 6: Exosomes: A Marker Resource that Identifies Cells of Origin

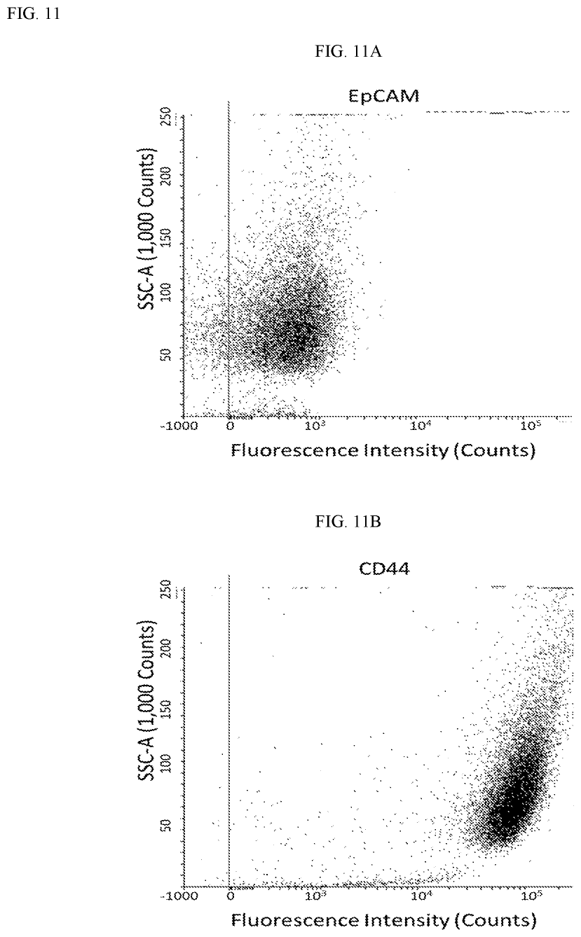

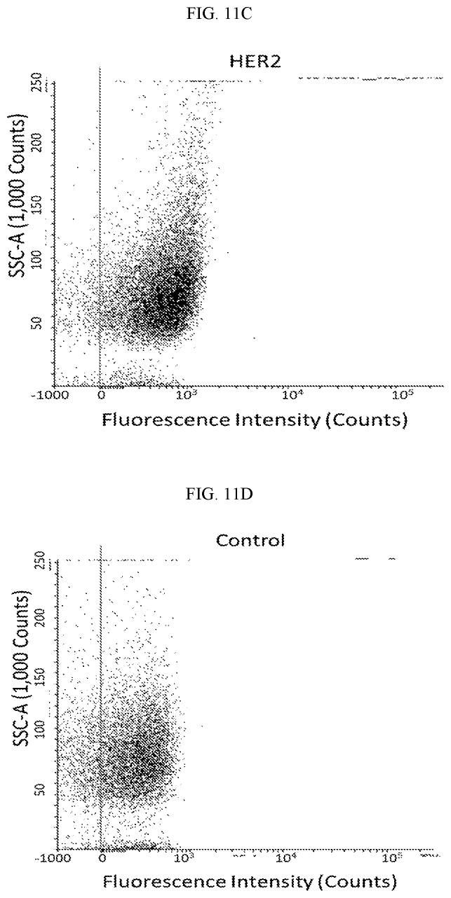

To investigate whether EXOs reflect their originating cells on biomarker expression, the expression of EpCAM, CD44, and HER2 was analyzed on the surface of MM231 cells via flow cytometry analysis. Phycoerythrin (PE)-conjugated antibodies and IgG were used for the fluorescent labeling and signal readout. The results show that the MM231 cells have very low expression of EpCAM and HER2, but high expression of CD44 (FIG. 11A, FIG. 11B, FIG. 11C, and FIG. 11D). MM231 cells are known to overpress (3+) CD44 with low expression (0-1+) of HER2 and EpCAM (C. Sheridan, et al. Breast Cancer Res. 2006, 8, R59; K. Subik et al. Breast cancer 2010, 4, 35; S. D. Soysal et. al. British J. Cancer 2013, 108,1480). Compared to the expression pattern of these three markers between EXOs and the originating cells, the experiments of this example found that EXOs reflect their originating cells on cancer biomarker expressions. The experiments of this example show that EXOs represent a biomarker resource for cancer detection.

### Example 7: Capture Raman Extracellular Vesicle Assay (cREVA)

Schematic illustrations of the methodology of the capture Raman Extracellular Assay (cREVA) are shown in FIG. 12A, FIG. 12B, and FIG. 12C. FIG. 12A and FIG. 12B show the principle of the assay and FIG. 12C shows the operation methodology when using the assay. The assay contains four sequential steps: (1) Antibody functionalization of the EV array device to target surface proteins of interests on EVs; (2) EV binding to the targeting antibodies; (3) EV labeling with the SERS AuNR tags; and (4) Signal collection with a portable and high-performance Raman spectrometer. EV labeling with SERS AuNR is based on electrostatic interactions between the negatively charged lipid membrane of EVs and the positively charged SERS AuNRs. The EV device has multiple wells that allow for analysis of different proteins or different EVs on the same device simultaneously. The array takes about 6 to 7 h. The method gives a quantitative measurement of the target surface proteins of interests on EVs and thus a quantitative surface protein expression profile of EVs. The results (i.e. the protein expression profile on EVs) can be used to understand EV biology, diagnose disease (e.g. cancer), monitor disease progression, and monitor patient treatment response.

The EV device is described in Example 1. The antibody functionalization is performed by incubating 50 μg/mL targeting-specific HS-PEG-Ab for 5 h at RT followed by incubation with 0.1 mM MU-TEG for 30 min at RT (FIG. 13). The surface is washed after each step with PBS to get rid of unbound molecules. EV binding is performed by incubating EV solution in the wells for 30 min at RT.

To label EVs, SERS AuNR tags are prepared by mixing 2 nM of AuNRs solution with 10 μM QSY21 for 15 min at RT (FIG. 14). The SERS AuNR tags are purified by centrifugation (14000 rpm, 10 min) and resuspended in PBS. The SERS AuNR tags are aged for 2 h before use and used within 5 h after preparation.

EV detection, signal collection, and data analyses follow the description in Example 1.

### Example 8: Target-Specific Capture of EXOs with cREVA

The cREVA specifically capture EVs based on the targeting proteins. FIG. 15 shows an example of specific EXO capture using the cREVA. MM231 EXOs were isolated from the continued culture supernatant via differential centrifugation. FIG. 15A shows the fluorescence image of captured MM231 EXOs using CD63 antibody as the capture ligand and FIG. 15B shows the fluorescence image of MM231 EXOs using IgG as the control ligand. MM231 EXOs were found with the CD63 antibody, but no with the IgG control protein, which indicating the high specificity of the cREVA for target-specific EXO capture.

### Example 9: Highly Specific and Sensitive Detection of EXOs with cREVA

The specificity and sensitivity of cREVA for EV detection was examined using MM231 EXO as the model EV and CD63 as the EXO marker. FIG. 16A shows the results on the specificity test. In the specificity studies, we have tested different surface modification of the device including (1) modification with MU-TEG only and no EXOs were incubated; (2) modification with MU-TEG only and EXOs were incubated; (3) Modification with MU-TEG and HS-PEG-CD63 antibody and no EXOs were incubated; (4) Modification with MU-TEG and HS-PEG-SH CD63 antibodies and EXOs were incubated; (5) modification with MU-TEG and HS-PEG-IgG protein, EXOs were incubated. All (1) and (5) were labeled with SERS AuNRs. While (4) showed strong signals, other treatments showed negligible signals. These results demonstrate that cREVA is highly specific to the target proteins of interest.

FIG. 16B shows the results on the sensitivity test via titration studies with a series of EXO dilutions. The results were presented as the dose-response curve using 11497 values at different EXO concentrations. As EXO concentration increased, the SERS signal intensity increased. Rapid signal increase was found above 1×10⁷/mL EXO concentration. At high concentration of EXOs (over 10⁹/mL), the signal leveled off due to the saturation of the antibodies grafted in the well of the device. Based on this titration studies, the LOD was determined to be 2×10⁶ EXOs/mL, which is 1000 times lower than that in human plasma. Based on the amount of EXOs added, the immobilization efficiency, the sizes of the well and the laser spot, we calculated that the signals were collected from 210 EXOs on the surface of Au slide in the well. This suggests that only about 200 EXOs are needed under the laser beam for detection.

Compared to dREVA, the cREVA is less sensitive, probably due to the limited amount of antibodies on the surface of Au slide. It takes 4 to 5 h longer than dREVA because of the elongated time on antibody binding on the Au slide.

### Example 10: Strong Correlation of cREVA with ELISA for EXO Protein Profiling

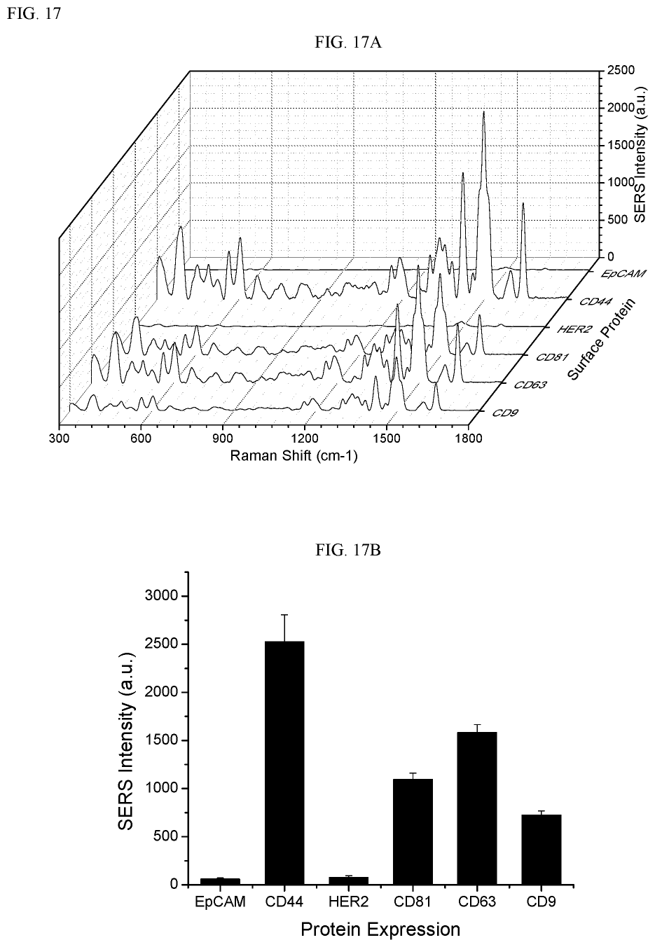

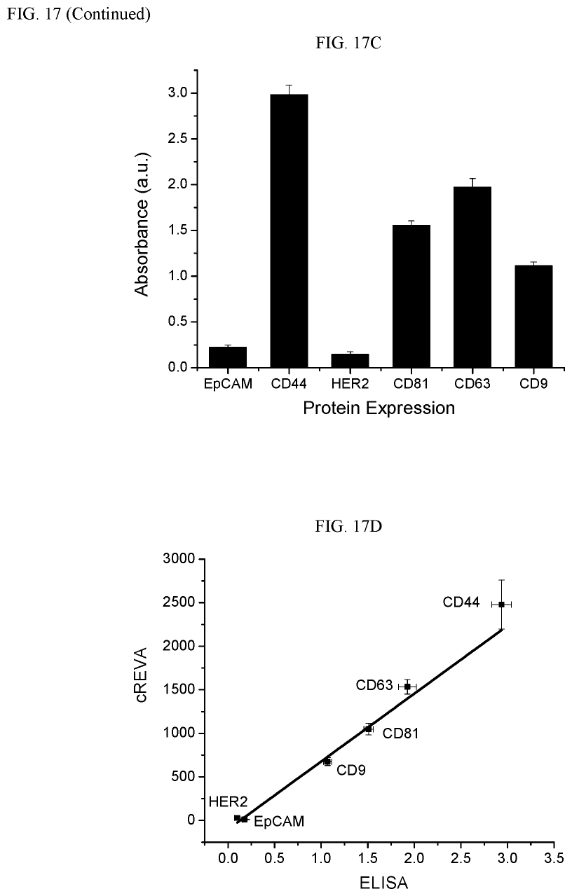

The ability of cREVA for EV protein profiling is tested and validated with traditional ELISA using MM231 EXO model. We analyzed six including one epithelial marker (EpCAM), two breast cancer markers (CD44 and HER2) and three EXO markers (CD81, CD63, and CD9). FIG. 17A and FIG. 17B show the averaged SERS spectrum for each target protein (n=3) and the expression profile of all six proteins on MM231 EXOs using the mean vale±SD from FIG. 17A respectively. The results show that MM231 EXOs have high expression of CD44 and the three exosome markers CD81, CD63 and CD9. They have very low expression of EpCAM and the other breast cancer marker HER2 (FIG. 17C). These results are consistant with results using dREVA.

The cREVA was validated using the gold standard ELISA. A quantitative comparison shows that our Raman assay has high correlation to ELISA, with correlation coefficient R² of 0.96 (FIG. 17D).

### Example 11: Application of cREVA for Detecting Cancer Markers on EXOs Derived from Different Breast Cancer Cell Lines

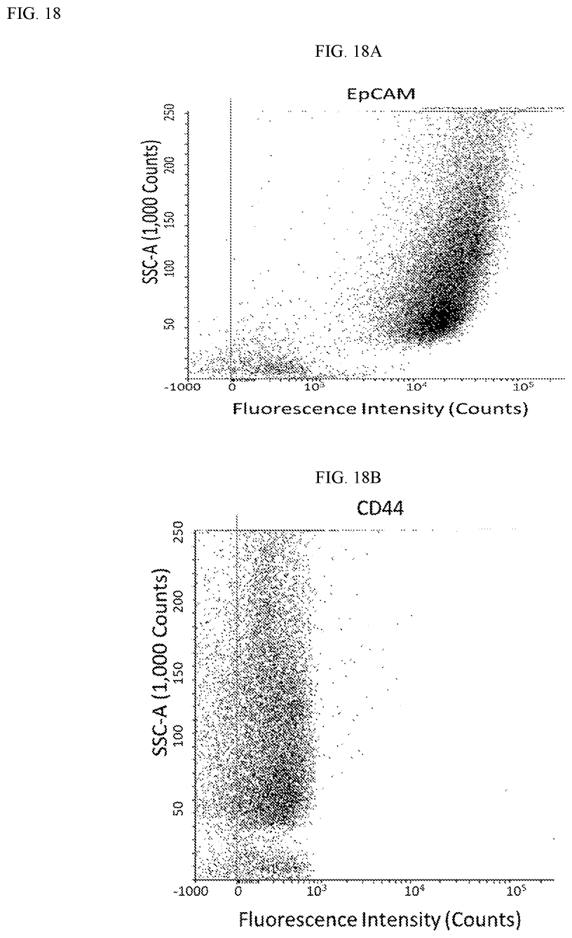

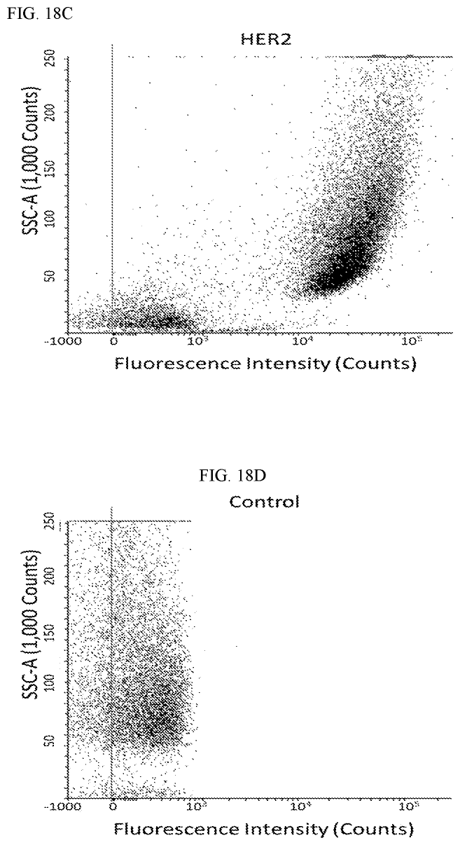

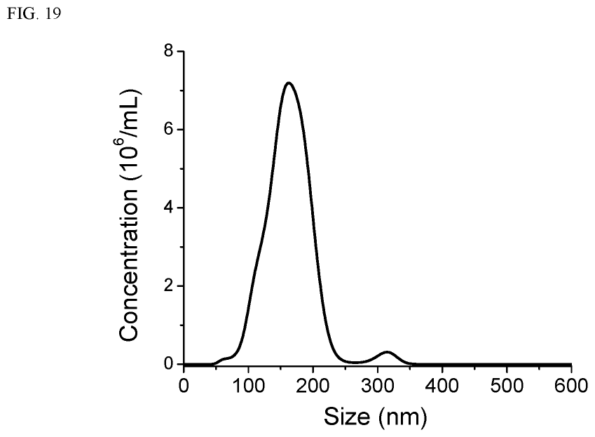

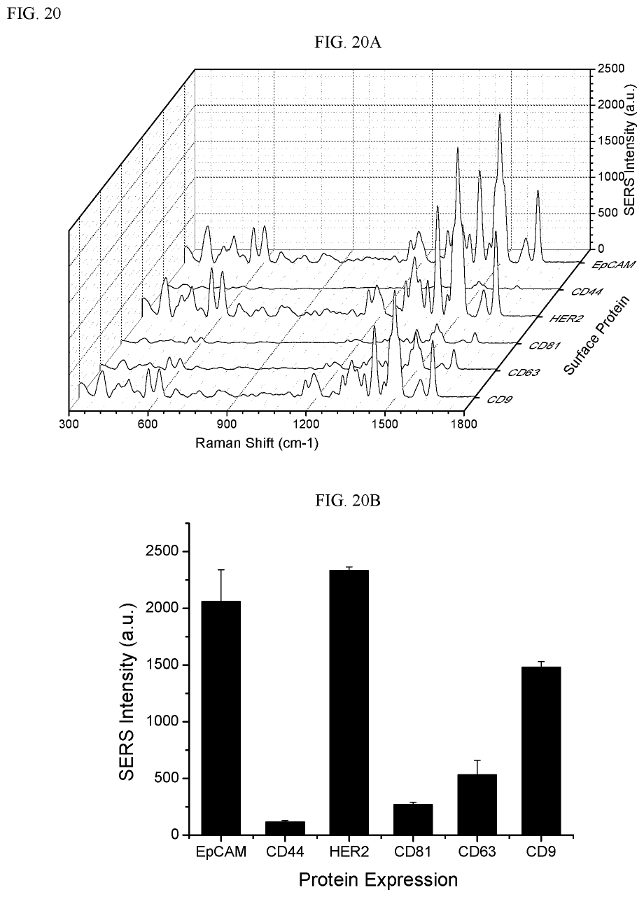

The cREVA has been tested to detect cancer markers on different cell lines. In these studies, we profiled EpCAM, CD44, HER2, CD81, CD63, and CD on breast cancer MM231 and SKBR3 and normal breast cells MCF12A. Flow cytometry analysis showed that SKBR3 cells have high expression EpCAM and HER2 and low expression of CD44 (FIG. 18A, FIG. 18B, FIG. 18C, and FIG. 18D). To analyze their EXOs, we isolated SKBR3 EXOs from conditioned culture supernatant with differential centrifugation. NTA shows that the SKBR3 cells were 165±38 nm (FIG. 19). FIG. 20A and FIG. 20B show the averaged SERS spectrum for each target protein (n=3) and the expression profile of all six proteins on SKBR3 EXOs using the mean vale±SD from FIG. 20A respectively. The results show that SKBR3 EXOs have high expression of EpCAM and HER2 and low expression of CD44. This protein pattern reflect the originating cells. Thus, we can detect the HER2 cancer markers on SKBR3 cells. As described in Example 10, we have detected the CD44 markers on MM231 EXOs. Thus, the cell-line-specific markers can be detected on their derived EXOs.

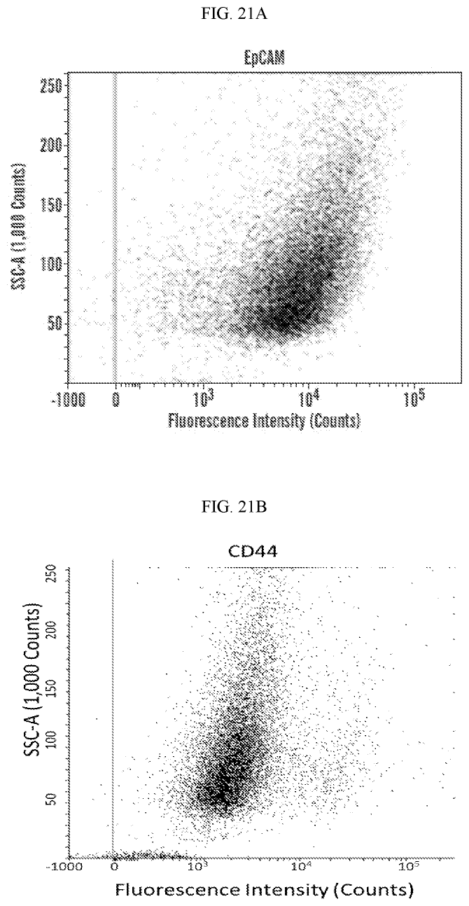

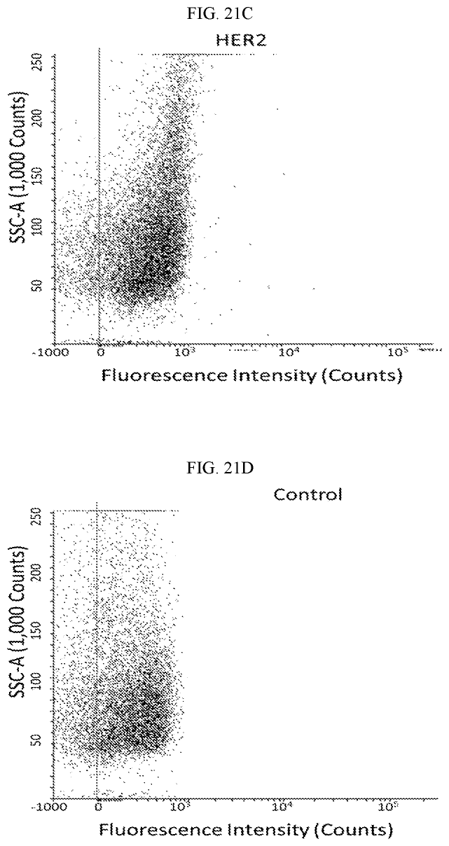

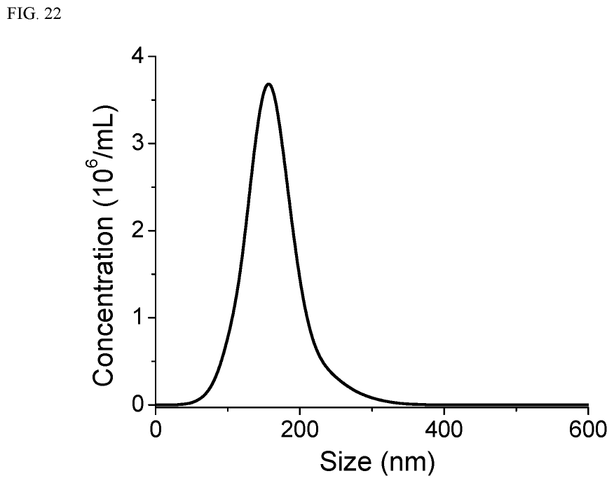

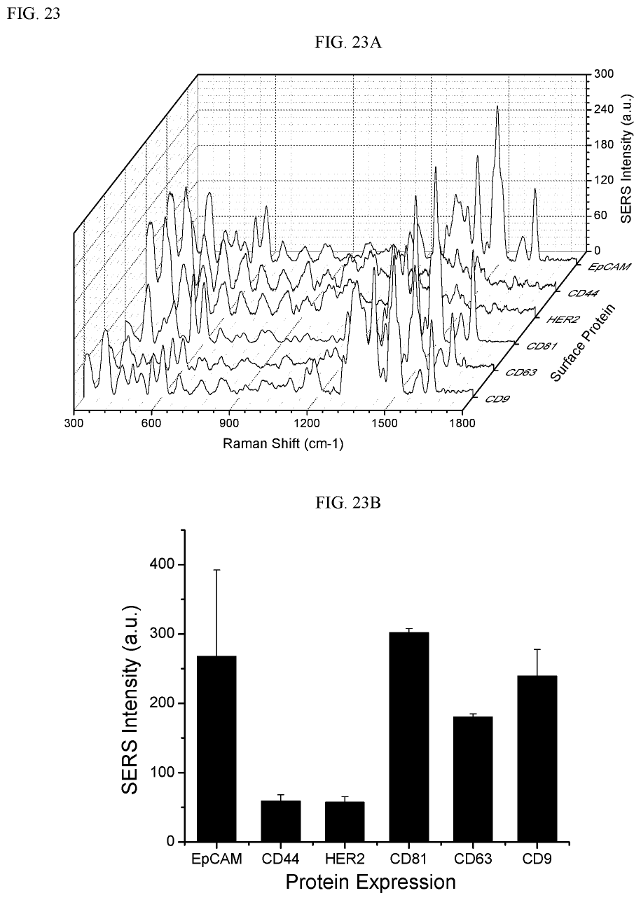

Flow cytometry analysis showed that the normal MCF12A cells positive for EpCAM and low expression of CD44 and HER2 (FIG. 21A, FIG. 21B, FIG. 21C, and FIG. 21D). Compared to SKBR3 cells, the MCF12A cells have much lower EpCAM expression. The MCF12A EXOs were 161±40 nm (FIG. 22). The results in FIG. 23A and FIG. 23B show that the EXOs are positive for EpCAM and low expression of CD44 and HER2. This protein pattern reflect that on the originating cells.

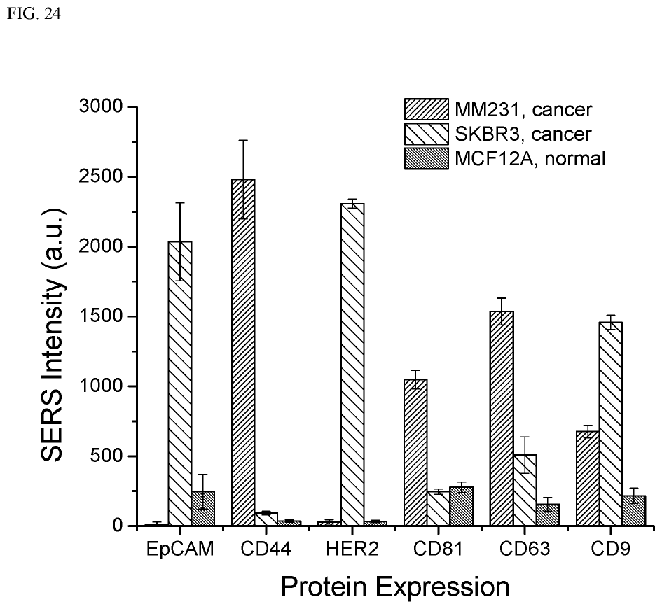

Using cREVA to analyze the surface markers on multiple cell lines, the experiments of this example have demonstrated that EXOs reflect their originating cells on surface protein marker expressions. The cancer-specific marker (CD44 for MM231 cells and HER2 for SKBR3 cells) are presented on cancer-derived EXOs, but not on normal cell-derived EXOs (FIG. 24). Thus, EXOs are a resource of protein biomarkers for diagnosis of cancer and potentially other diseases.

### Example 12: Application of cREVA for Detecting Cancer Markers on EXOs from Breast Cancer Patients

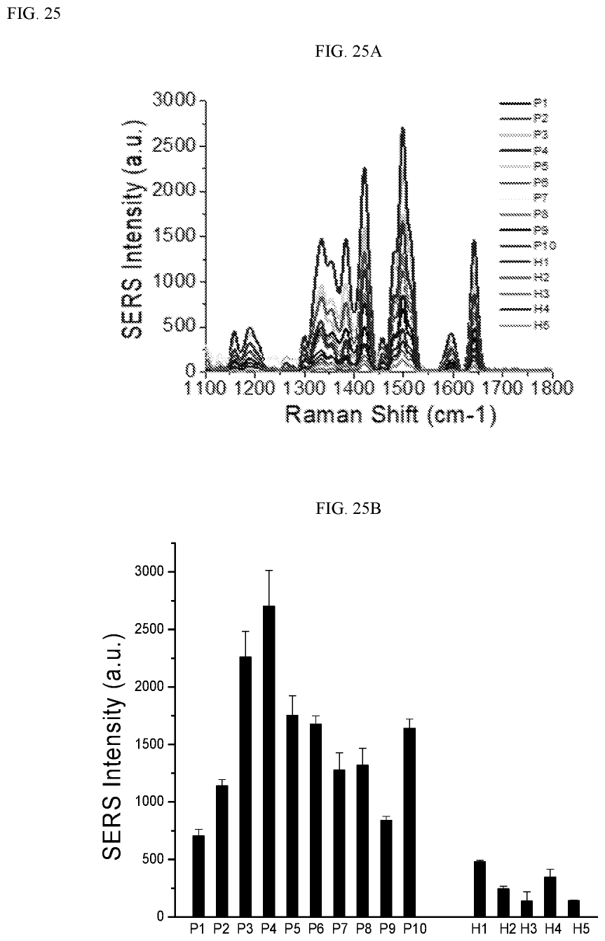

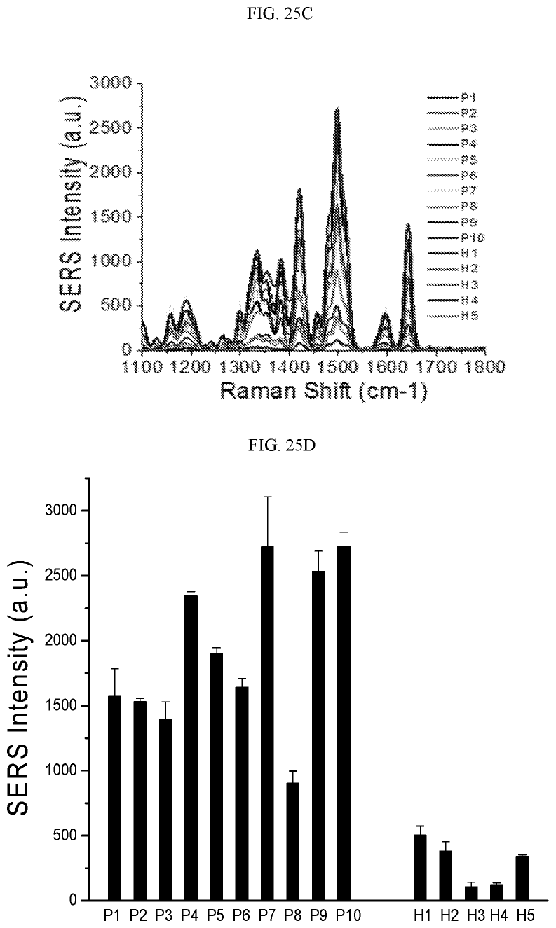

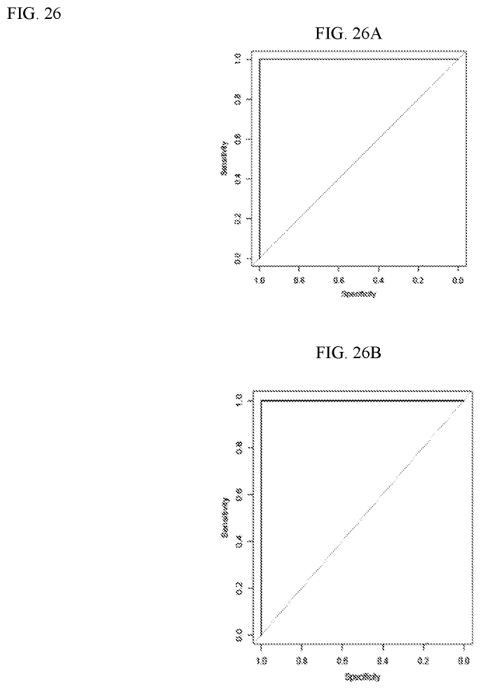

The cREVA has been tested for breast cancer diagnostics. Due to the heterogeneous breast cancer types, we chose HER2-positive patients (n=10) for a proof-of-concept study. The disease includes invasive lobular carcinoma, infiltrating ductal carcinoma, and adenocarcinoma of the breast in stages I, II, and III. We obtained patient plasma samples from the XpressBank at Asterand Bioscience. To collect plasma samples from healthy donors (n=5), we obtained fresh whole blood and extracted exosomes by differential centrifugation. By profiling different proteins, we found EpCAM and HER2 are biomarkers to distinguish breast cancer patients from normal controls. As shown in FIG. 25, the levels of EpCAM and HER2 were significantly higher in the tested breast cancer patient samples than in the control groups (p<0.01 for both markers). Specifically, FIG. 25A shows average SERS spectra (n=3) from each subject for the EpCAM marker. FIG. 25B shows the protein expression profiles based on the data in FIG. 25A. The p-value between cancer patients and healthy donors for EpCAM is 7.4×10⁻¹¹. FIG. 25C shows average SERS spectra (n=3) from each subject for the HER2 marker. FIG. 25D shows the protein expression profiles based on the data in FIG. 25C. The p-value between cancer patients and healthy donors for HER2 is <2.2×10⁻¹⁶. Our finding of HER2 marker (AUC=1 from ROC curve, FIG. 26A) on exosomes in the HER2-positive breast cancer patient is consistent with previous studies with a SPR method (A. A. I. Sina et al., Sci. Rep. 2016, 6, 30460). In addition, we identified EpCAM as another biomarker to differentiate exosomes from breast cancer patients from normal controls (AUC=1, FIG. 26B). EpCAM has been previously identified as an exosome-based biomarker for ovarian cancer in ascites samples (Im et al., Nat. Biotechnol. 2014, 32, 490). Here we report EpCAM as an exosome-based biomarker for breast cancer. The early promise of these proteins for breast cancer diagnosis, however, requires further validation with larger cohorts.

In conclusion, this aspect of the present disclosure provides a simple, rapid, inexpensive, highly sensitive, and highly specific Raman-based assay for point-of-care detection and molecular profiling of EVs and other membrane bound vesicles. The assay can be performed in two ways, direct Raman extracellular assay (dREVA) and capture Raman extracellular assay (cREVA). Using the assays (both dREVA and cREVA) and model EXOs from breast cancer cells, the experiments of the preceding examples showed that EXOs express cancer markers in a similar pattern to their donor cancer cells, suggesting the potential use of screening EXOs for biomarkers for cancer detection and investigation. The assay can be widely used for basic and clinical cancer research.

The dREVA can be technically modified for automatic and high throughput clinical test of large scale of samples in real-time by using an EV microarray platform. The EXOs can be directly deposited onto the lipophilic Au slide with pico- to lower nanoliter EVs using the well-established high speed and high throughput microdrop printing technology. The microdrop printing can make over 800 EV spots on the micrometer size scale on one Au slide. This next generation REVA has the potential to revolutionize EV research and realize a novel cancer liquid biopsy approach for cancer research and diagnosis.

The results described herein above, were obtained using the following methods and materials.

### Materials

All chemicals were purchased from Sigma-Aldrich unless specified. Antibodies were purchased from Biolegend (San Diego, Calif.). QSY21 carboxylic acid-succinimidyl ester was purchased from Thermo Fisher Scientific. PE-labeled antibodies were purchased from Miltenyi Biotec (Auburn, Calif.). All cell lines were purchased from ATCC (Manassas, Va.). Cell culture media were purchased from VWR (Radnor, Pa.) and fetal bovine serum (FBS) was purchased from Fisher Scientific (Waltham, Mass.).

### Synthesis of Small Gold Nanorods (Au NRs)

Au NRs were synthesized in two steps: preparation of Au seeds and growth of Au seeds into AuNRs in a growth solution. To make the Au seed solution, 0.5 mL of 1 mM chloroauric acid (HAuCl₄) was added to 1.5 mL of 0.13 M cetyltrimethylammonium bromide (CTAB) solution with constant stirring. 120 μL of 10 mM ice-cold sodium borohydride (NaBH₄) was quickly injected and the solution was stirred for 3 min to form the Au seed solution. The Au seed solution was kept undisturbed for 3 hours in 25° C. water bath before its use. In a different glass vial, 5 ml of 1 mM HAuCl₄ was added 5 mL of 0.2 M CTAB solution followed by addition of 125 μl of 4 mM silver nitrate (AgNO₃). After mixing with stirring, 12 μl of Au seed solution was quickly injected into the solution and left undisturbed for 10 min to form small AuNRs. The solution was centrifuged at 14000 rpm for 10 min and the AuNR pellet was resuspended with ultrapure water for further use.

### Preparation of Target-Specific Antibody-Conjugated SERS AuNR Tags

To a 0.25 mL of 1 nM AuNR solution, 10 μL of 25 μM HS-PEG-Ab was added and gently stirred for 5 h at RT. Then 25 μL of 100 μM QSY21 carboxylic acid (hydrolyzed from QSY21 carboxylic acid-succinimidyl ester) was added and stirred for 15 min at RT. At last, 25 μL of 1 mM MHDA-PEG was added and stirred for 1 h at RT. The solution was centrifuged at 14,000 rpm for 10 min to precipitate down the antibody-conjugated SERS AuNR tags. The HS-PEG-Ab was prepared in advance by reacting 10 μL of 1 mg/mL antibodies with 10 μL of 1 mM HS-PEG-NHS MW 5000 in PBS for overnight at 4° C. After reaction, the free HS-PEG-NHS was separated by membrane filtration with a 10 KD Nanosep filter (PALL Life Sciences).

Preparation of SERS AuNR tags

100 μL of 100 μM QSY21 carboxylic acid aqueous solution was added to 1 mL of 2 nM AuNRs and the mixture was stirred for 15 min at RT. After purification by centrifugation (14000 rpm, 10 min), the SERS AuNR tags were resuspended in PBS to make 1 nM solution. The solution was aged at room temperature (RT) for 2 h before use.

### Au Thin Film Deposition on Microscopic Glass Slide

A standard microscopy glass slide (75×25×1 mm) was coated with 10 nm thick Au film by magnetron sputtering technique using an ORION-AJA system from a 99.99% pure Au target. The deposition of the Au layer was performed on a 4 nm titanium layer previously deposited from a 99.99% pure titanium target on the glass slide. The slide-target distance was kept at 15 cm during the process. The film thickness was controlled by an INFICON SQM-160 quartz crystal monitor/controller equipment. The rotating substrate-holder was kept at 80 rpm. The films were grown in an atmosphere of argon at 3.0 mTorr and a gas flow of 15 sccm, with the DC power supply set to 100 W and the pressure before inserting the argon was 4.0×10⁻⁸ Torr. The whole process took 4 h.

### Fabrication of Array Template

Plastic (polylactic acid) array templates with specified well size and inter-well distance were fabricated using a MakerBot Replicator PC 3D printer. The template was attached with a rubber array via a layer of glue composed of 60% silicone and 40% mineral spirit. This rubber array was made from 1.6 mm thick rubber sheet in the same dimensions as the template via punctuation. The assembled plastic and rubber arrays were used as a template array to make antibody array on the Au-coated glass slides.

### Fabrication of Array Template

Plastic (polylactic acid) array templates with specified well size and inter-well distance were fabricated using a MakerBot Replicator PC 3D printer. The template was attached with a rubber array via a layer of glue composed of 60% silicone and 40% mineral spirit. This rubber array was made from 1.6 mm thick rubber sheet in the same dimensions as the template. The rubber was punctured with 2 mm 0 perforations to make the array. The assembled plastic/rubber array was used to make EV array on the Au-coated glass slide.

### Lipophilic Coating of the EV Array Device

The template array was attached onto the surface of the Au-coated glass slide with ¾″ wide heavy-duty binder clips. Into each well, 20 μL of 1 mM DSPE-PEG-SH was added and incubated for 1 h at RT. Then, 5 μL of 0.5 mM MU-TEG was added and incubated for 30 min at RT. The unbound chemicals were removed by washing three times with PBS.

### Antibody Functionalization of the EV Array Device

The template array was attached onto the surface of the Au-coated glass slide with ¾″ wide heavy-duty binder clips. 25 μL of 50 μg/mL target-specific antibody-linked HS-PEG-Ab in PBS was added into the wells and incubated for 5 h at RT. The antibody-treated wells were washed for three times with PBST (100 mL PBS+0.5 mL Tween 20 (0.5%)) to get rid of unbound proteins. Then, 15 μL of 0.1 mM MU-TEG was added into the wells and incubated for 30 min at RT to saturate the Au surface. The antibody-functionalized wells were washed three times with PBST and stored at 4° C. for further use. Isotype IgG was used as the negative control.

### Cell Culture

Human breast MDA-MB-231 (MM231) cancer cells were cultured in DMEM with high glucose with 10% fetal bovine serum (FBS) at 37° C. under 5% CO₂. Human breast SKBR3 cancer cells were cultured in RPMI 1640 medium with 10% fetal bovine serum (FBS) at 37° C. under 5% CO₂. Human breast normal cells MCF12A (immortalized) were cultured in DMEM/F-12 medium with 5% fetal horse serum, 1% Pen/Strep (100×), 0.5 mg/mL hydrocortisone, 10 μg/mL bovine insulin, 100 ng/mL cholera toxin, 20 ng/mL EGF.

### Isolation and Characterization of EXOs in Culture Media

Cells were grown in conditioned cell culture media (media+10% exosome-free FBS) for 48 h. The EXO-free FBS was obtained by separating EXOs from FBS with two times of ultracentrifugation (100,000 g, 70 min). To collect EXOs, the conditioned cell culture supernatant was collected and centrifuged at 430 g at RT for 10 min. The supernatant was collected and centrifuged at 16,500 g at 4° C. for 20 min. The supernatant was collected and centrifuged at 100,000 g at 4° C. for 70 min. After removing supernatant, the exosome pellet was resuspended in cold sterile PBS and centrifuged again at 100,000 g at 4° C. for 70 min. The exosome pellet was resuspended in cold sterile PBS, filtered with a 0.20 μm filtered with a 0.2 μm PES filter (Agilent Technologies), and stored at −80° C. before use. The concentration and size distribution of exosomes were characterized using NTA with a NanoSight LM10 microscope (Malvern Instruments, Inc).

Exosome Immobilization on the Lipophilic EV Array Device, Fluoresce Imaging and Labeling with the Target-Specific SERS AuNRs

25 μL of 6.25×10⁷/mL EXOs were added to the lipophilic Au array wells and incubated for 30 min at RT. After washing the wells three times with PBS, EXOs were labeled with 1 mM 3,3′ Dioctadecyloxacrbocyanine perchlorate (DiO) in PBS for 15 min at RT. EXOs were then washed with PBS and examined by a fluorescent microscope (Olympus IX 71) with a Prior Lumen 200 illumination system. The excitation was 482/35 nm and emission was 536/40 nm. For labeling with SERS AuNRs, 25 μL of 1 nM target-specific antibody-conjugated SERS AuNR tags were added and incubated for 30 min at RT. The wells were washed three times with PBS and immersed in 20 μL PBS for detection.

Exosome Binding on the Antibody-Functionalized EV Array Device, Fluoresce Imaging, and Labeling with SERS AuNRs

25 μL of 6.25×10⁷/mL EXOs were added to the antibody-functionalized Au array wells and incubated for 30 min at RT. After washing the wells three times with PBS, EXOs were labeled with 1 mM DiO in PBS for 15 min at RT. EXOs were then washed with PBS and examined by a fluorescent microscope (Olympus IX 71) with a Prior Lumen 200 illumination system. The excitation was 482/35 nm and emission was 536/40 nm. For labeling with SERS AuNRs, 25 μL of 1 nM SERS AuNR tags were added into each well and incubated for 30 min at RT. The wells were washed three times with PBS and immersed in 20 μL PBS for detection.

### Signal Collection and Data Analysis

Raman signals were collected with a TSI ProRaman spectrometer (X=785 nm). The laser beam size at focus was 200 μm. Each spectrum was collected with the laser power of 50 mW and acquisition time of 1 s. A baseline correction using a multi-segment polynomial fitting was automatically performed by the signal acquisition software (EZRaman Reader v8.1.8) to subtract SERS background (broad continuum emission). The peak at 1497 cm⁻1, which is the strongest one among all the peaks of the QSY21 SERS spectrum, was used as the representative peak for analysis. To account for the variations from instrumentation response and batch-to-batch nanotag preparation, the spectrum of the SERS nanotag solution (0.1 nM) during each experiment was collected and the intensity of the 1497 cm⁻¹ peak was normalized to 2000 a.u., the typical value of a 0.1 nM nanotag solution. This gave a correction factor for each nanotag to correct the signal intensity from EXOs labeled with that nanotag during each experiment. The corrected intensity of the 1497 cm⁻¹ peak was used for analysis.

### Enzyme-Linked Immunosorbent Assay (ELISA)

50 μl of 6.25×10⁸/mL MM231 EXOs were added into 96-well polystyrene plate (Corning Incorporated) wells and incubated at 4° C. for overnight. The wells were washed three times with Dulbecco's phosphate-buffered saline (DPBS) followed by incubation with 100 μl of blocking solution (DPBS with 4% BSA) at RT for 2.0 h. After washing three times with DPBS, each well was treated with the following solutions subsequently, 50 μL of 2 μg/ml target-specific antibodies (2 h, RT), 50 μl of HRP-conjugated anti-mouse IgG antibody (ThermoFisher, 1:60 dilution in blocking solution) (1 h, RT), and 100 μl of 3,3,5,5-tetramethylbenzidine solution (TMB, Sigma-Aldrich) (30 min, RT). The wells were washed three times with DBPS between steps. After the TMB incubation, 100 μl of 2 M sulfuric acid (H₂SO₄) was added to stop the reaction. The optical density of each well was measured at 450 nm using a BioTEK ELx800 absorbance microplate reader. Isotype IgG was used as the control.

### Example 13: Detection and Analysis of Single Vesicles

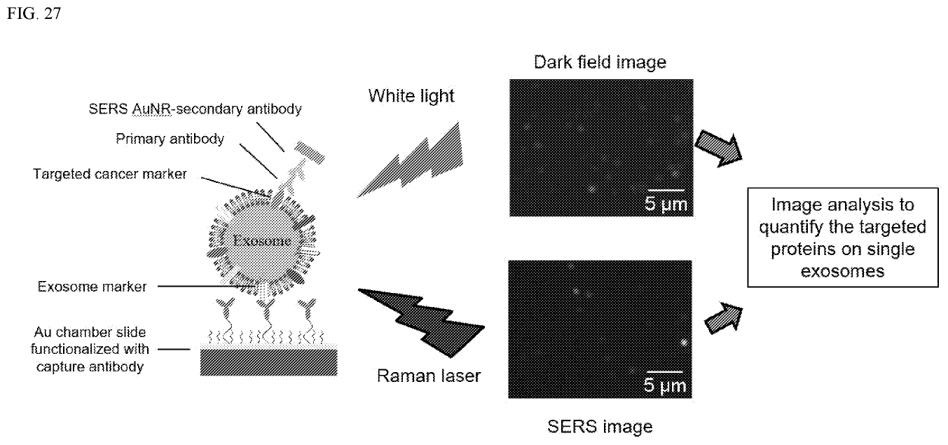

One aspect of the present disclosure describes methodologies for protein profiling of membrane-bound single vesicles focusing on exosomes (EXOs) using SERS imaging with SERS nanotags as contrast agent (SERS-Single Vesicle Technology or SERS-SVT). FIG. 27 provides an overview of the method, in which exosomes are captured on a gold (Au)-coated surface via anti-CD81 antibodies. Targeted proteins of interests are recognized with primary antibody and then SERS AuNR-conjugated secondary antibody. Exosomes are then imaged under dark field to localize exosomes, and targeted proteins on exosomes are detected by Raman imaging. By imaging analysis of the dark field image (called mask image) and the Raman image (called target image), the protein on each exosome can be quantified to give the expression profile of the targeted protein marker on exosomes under investigation.

### Fabrication of a Multi-Well Chamber Slide

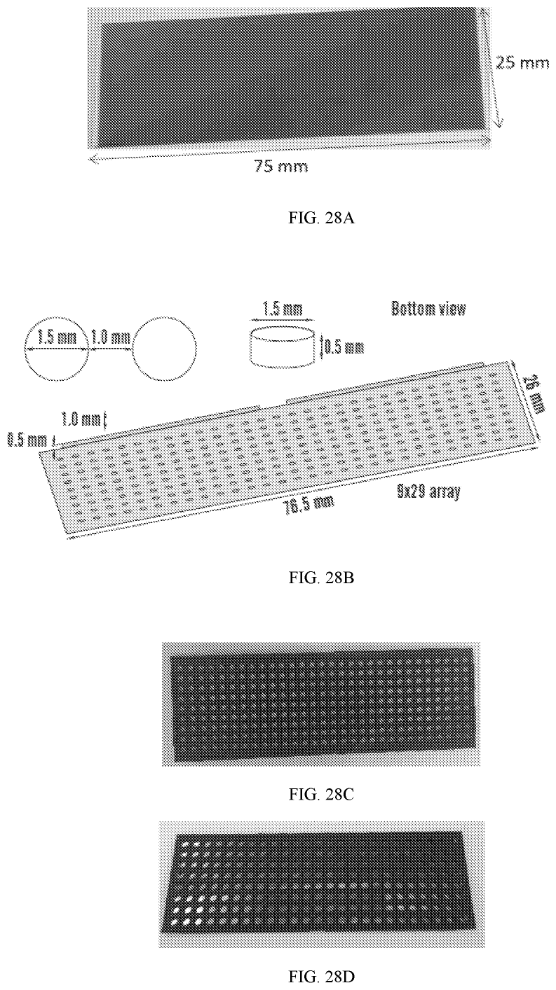

EXOs are captured and analyzed on Au-coated standard microscope glass slide (75×25×1 mm) (FIG. 28A). The Au film, 100 nm in thickness, is used to facilitate surface modification. It is coated onto the glass slide using a AJA sputter system. To improve sample throughput and minimize reagent consumption, we designed a multi-well cassette (FIGS. 28B and 28C). The cassette contains 200 (8×25) wells, with the diameter (D), spacing (d), and height (H) of each well being 2.0, 1.0, and 0.5 mm, respectively. It is designed with stabilizers on the edges to help fixation to the Au slide to from a chamber slide (FIG. 28D). Sealing is assisted with pressure grease. The cassette can be removed, washed, and reused without damage. It was fabricated with a Formlabs Form 2 3D printer. Each well of the chamber slide holds maximally 1.5 μl solution, with a typical working volume of 1.0 μl.

Direct Capture of EXOs from Biofluids

Tetraspanin CD81 is an EXO marker that differentiates EXOs from other types of extracellular vesicles; therefore, CD81 antibody was used to capture EXOs from biofluid. The method can directly capture EXOs with CD81 monoclonal antibodies from plasma and other biofluids without EXO pre-purification. The CD81 antibody was conjugated to a polyethylene glycol thiol (PEG-SH) linker (MW=5000) by reacting HS-PEG-NHS with CD81 antibody (100:1 molar ratio) at 4° C. for overnight and then purified by filtration centrifugation. EXOs were diluted in conditioned cell culture medium (cell culture medium without fetal bovine serum) with phosphate buffer solution (PBS) and filter with 0.2 micron membrane filter.

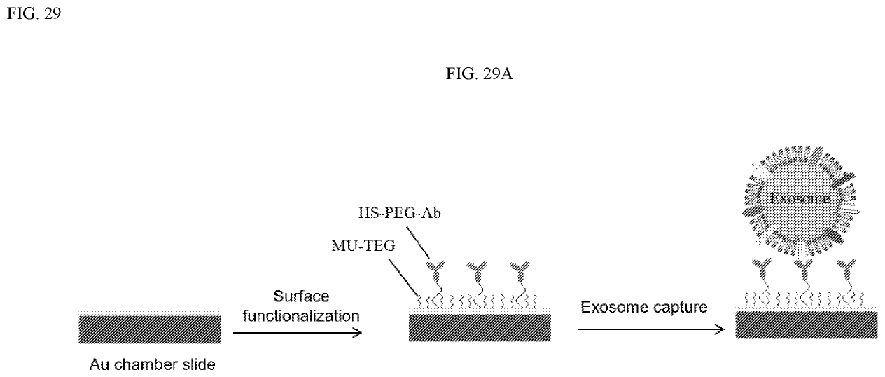

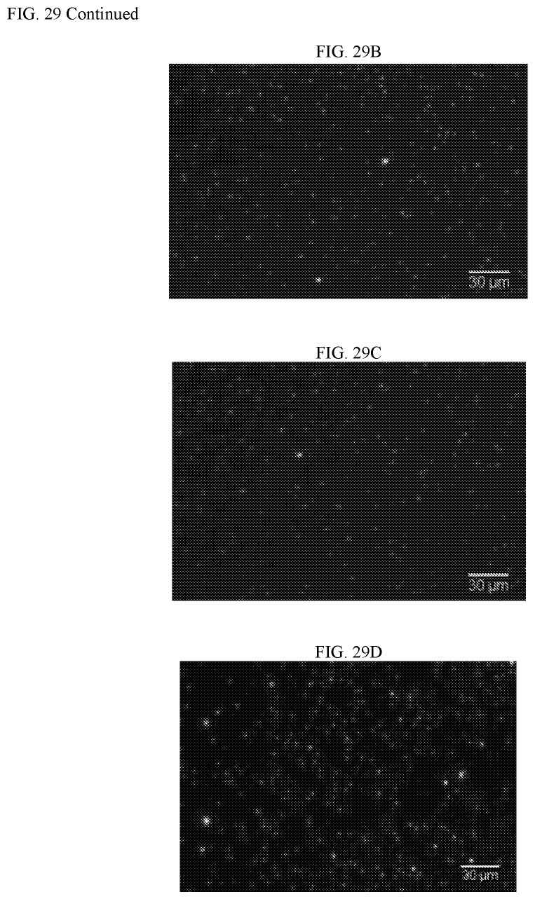

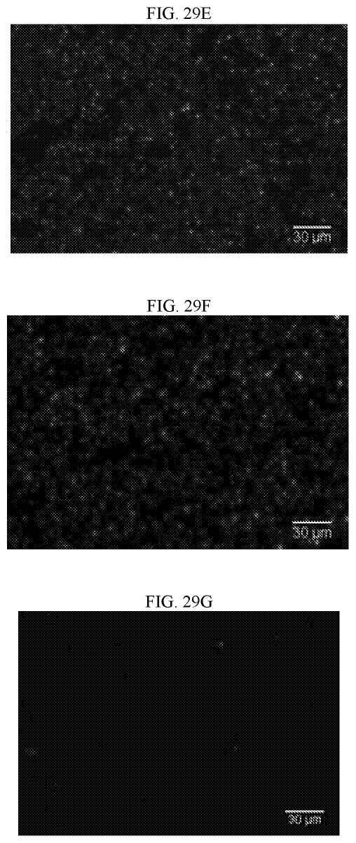

The procedure used to capture EXOs from plasma included the following steps (FIG. 29A): (1) Contacting a chamber slide having an Au surface with a CD81 antibody linked with PEG-SH (50 μg/mL) for about 5 hours at room temperature (RT), then washing the chamber slide with PBS; (2) Contacting the Au surface with 0.1 mM 11-mercaptoundecyl tetra (ethylene glycol) (MU-TEG) for 30 min at RT to saturate the Au surface, followed by washing with PBS; and (3) Incubating the EXO sample on the device for about 2 hours and then washing with PBS. As a control, an IgG polypeptide was used as the capture agent rather than an exosome specific marker.

FIGS. 29B to 29F show fluorescence images of captured exosomes with CD81 antibodies from MM231 EXOs (B) and SKBR3 EXOs in conditioned cell culture medium and from plasma samples of three different breast cancer patients (D-F). FIG. 29G shows the fluorescence image of captured EXOs from the patient of FIG. 29F with IgG control. The results show that EXOs can be specifically captured from either cell culture medium or plasma without time consuming purification.

Preparation of SERS AuNR tagged secondary antibodies was performed as described supra.

### Exosome Labeling

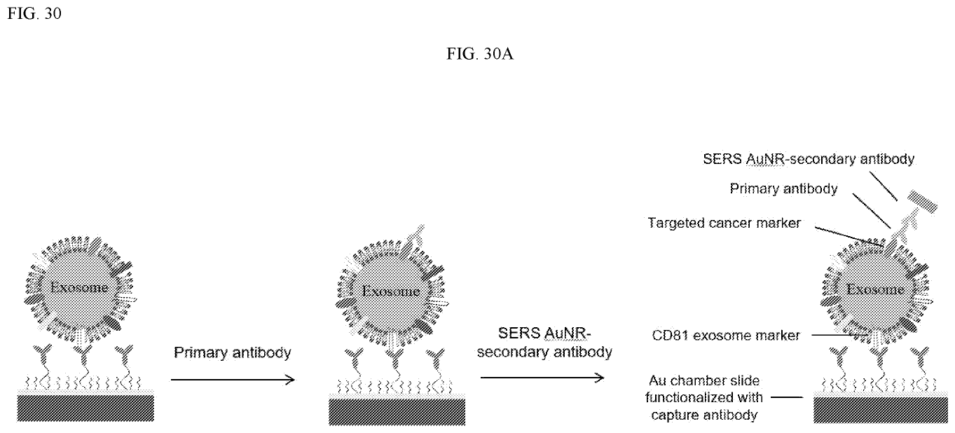

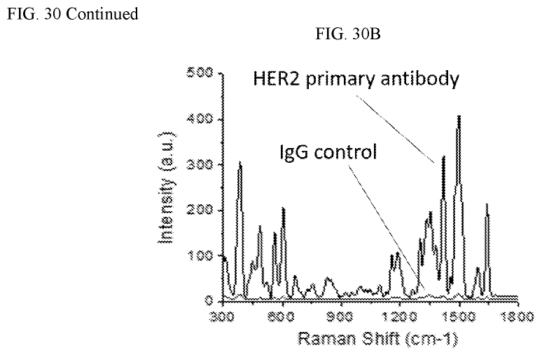

For specific protein detection on exosomes, an indirect assay was used (FIG. 30A). First, targeted proteins were labeled with anti-mouse primary antibody by incubation with 2 μg/mL of mouse anti-HER2 monoclonal antibody at RT for 2 h. Then, the primary antibody was incubated with 1 nM of SERS AuNR-secondary antibody for 1 hour at RT. This labeling was highly specific to the targeted antibodies. Anti-mouse IgG (H+L) highly cross-adsorbed secondary antibodies were used to minimize nonspecific interactions of the AuNRs with the capture antibody. FIG. 30B shows the SERS spectrum of HER2-targeted SKBR3 EXOs compared to the IgG control. While the IgG control gave signals at the background level (18 a.u. at the representative 1497 cm-1 peak), the targeted exosomes gave intense SERS signals with 1497 cm-1 of 443 a.u. This demonstrates that the labeling is highly specific and can reliably analyze targeted cancer protein markers.

### Instrumentation of SERS Imaging and Spectroscopic System

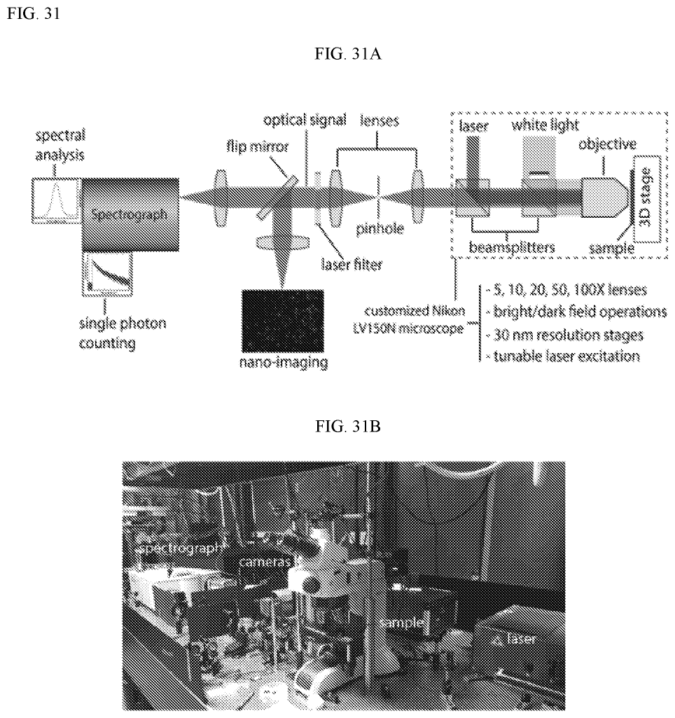

EXOs can be detected using a commercial Raman microscope with dark field modality. Alternatively, a versatile optical microscopic system for single EXO SERS analysis was developed by integrating an optical microscope (Nikon, LV 150N) with an excitation laser and confocal micro-Raman setup. FIG. 31A is a detailed schematic of the instrumentation. FIG. 31B is an image of the optical setup that we have developed and is available for this project. In this system, a modified Nikon LV 150N microscope with bright/dark field modes is used for optical excitation and detection. Chamber slide with EXO samples is mounted on a 3D nanometer-resolution translation stages (Newport, model 9063) and be illuminated by a halogen white light source (grey path) for bright/dark field observations.

For Raman measurements, the samples were excited by a Melles Griot continuous-wave He laser (Model 05-LPH-925) with a wavelength of 632.8 nm (maximum power: 35 mW) through an objective lens. The laser beam was defocused by a separate lens so a large area of the sample (170 um in diameter) can be homogenously illuminated. Reflected Raman signal, after passing through the beam splitters, was filtered by a long-pass filter (to block the laser excitation) and refocused onto an intermediate image plane. The Raman signals were detected by a Photometrics CoolSnap camera for nano-imaging. The Raman signals can also be collected by a spectrometer (Horiba Jobin Yvon, model iHR550) and detected by another charged-coupled-device (CCD) camera (Horiba Jobin Yvon, model Synapse) for spectroscopic analysis. The spectrometer and CCD camera were optimized for the visible frequency with up to 95% quantum efficiency capable of single exosome measurement. The system was fully automated by a set of Labview computer programs which synchronize all optical measurements. Thus, the same area of exosome samples on the chamber slide can be simultaneously detected with dark field light scattering imaging, Raman imaging, and Raman spectroscopy.

### Data Collection and Analysis

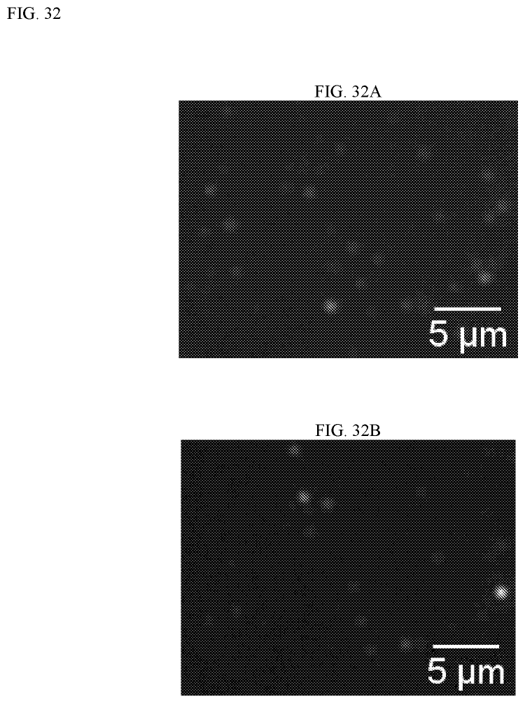

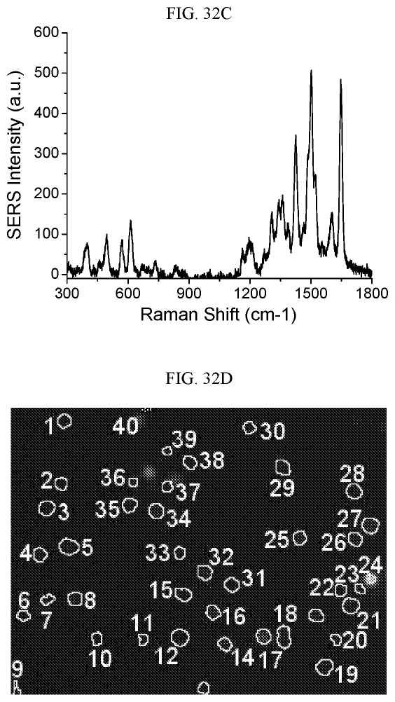

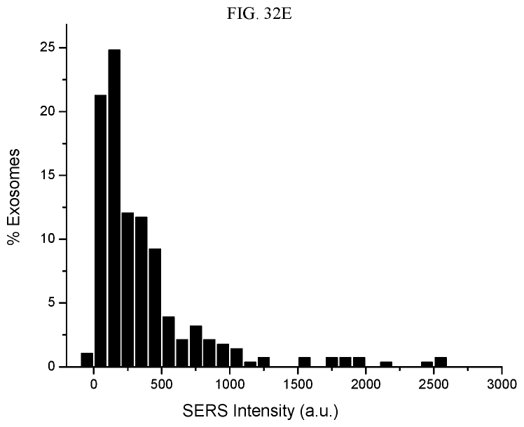

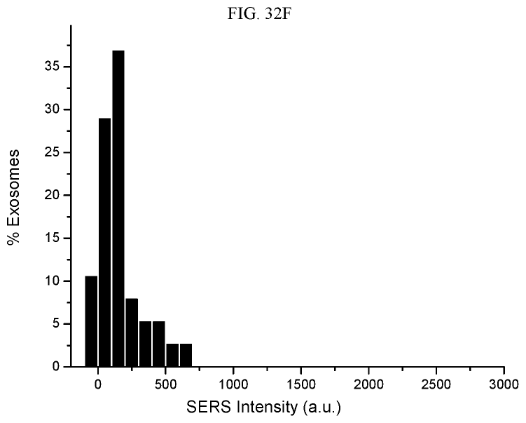

Data collection. FIGS. 32A to 32C shows an example of data collection by detecting CD44 on MM231 exosomes that are known to have high expression of CD44. Firstly, a dark field image was acquired with a 100× objective (WD=1 mm, air immersion) based on the strong light scattering properties of EXOs. This image served as the mask to localize EXOs (FIG. 32A). Then, the microscope was switched to Raman mode and SERS signals from EXOs were excited with the He laser at 632.8 nm. FIG. 32B shows the SERS image of MM231 exosomes with SERS AuNR labeling of the CD44 cancer marker (laser power: 1.5 mW. exposure time: 3 s). The CD44-positive exosomes show up in the SERS imaging mode, with brightness correlating to the expression level of CD44. Lastly, the SERS signals were confirmed via spectroscopic detection using the Raman spectrometer (FIG. 32C).

Data analysis. FIGS. 32D to 32H is an example of data analysis using data from FIGS. 32A and 32B. Image J was used to analyze the EXOs, wherein the mask image was first uploaded (32A). After a series of steps to adjust brightness, contrast, bandpass filter, and threshold, the outline of each EXO was created on the mask image. Then the target image (FIG. 32B) was added to the outlined mask using the ROI manager (FIG. 32D) to form an overlay image with the target image (FIG. 32E). The mean pixel intensity of the outlined areas of this overlay image was then extracted. The values were subtracted with the background from a blank neighboring area (noise) to give corrected signal intensities. By analyzing a number of exosomes, a histogram was generated that shows the distribution of the pixel intensity among all examined exosomes (FIG. 32F). This population density histogram represents the expression profile of the targeted protein on single EXOs. Using the same method, a control was obtained when the primary CD44 antibody was replaced with isotype IgG (FIG. 32G).

The targeted protein on an EXO was define as positive based on the cut off value from the IgG control. Three parameters were used define to measure the expression of a targeted protein p: fraction of the positive exosomes Fp, mean value of the protein level per EXO from the positive EXOs ζp, and mean value of the protein level per EXO from the total EXOs ζt. As reports an average value from all investigated EXOs, it is comparable to bulk measurement. In the example shown in FIG. 32 for CD44 on MM231 EXOs, FCD44, ζCD44, and ζCD44, t is 0.66, 5.7×105 a.u. and 4.0×105 a.u. respectively.

To account for the variations from batch-to-batch nanotags, the SERS spectrum of the nanotag solution (0.1 nM) before use was measured and the 1497 cm-1 peak was normalized to 2000 a.u., the typical value of a 0.1 nM nanotag solution. This gives a correction factor for each batch of nanotags. In the above data, the 1497 cm-1 for the 0.1 nM nanotag was 2010 a.u., therefore, correction was not needed in this study.

Profiling of HER 2 Expression on EXOs Derived from SKBR3 Cells

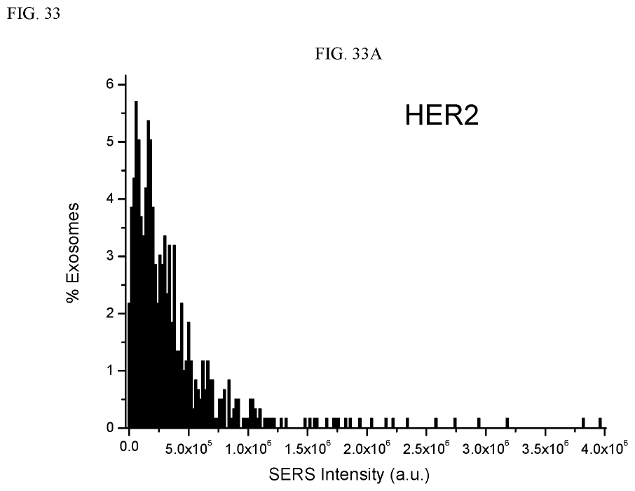

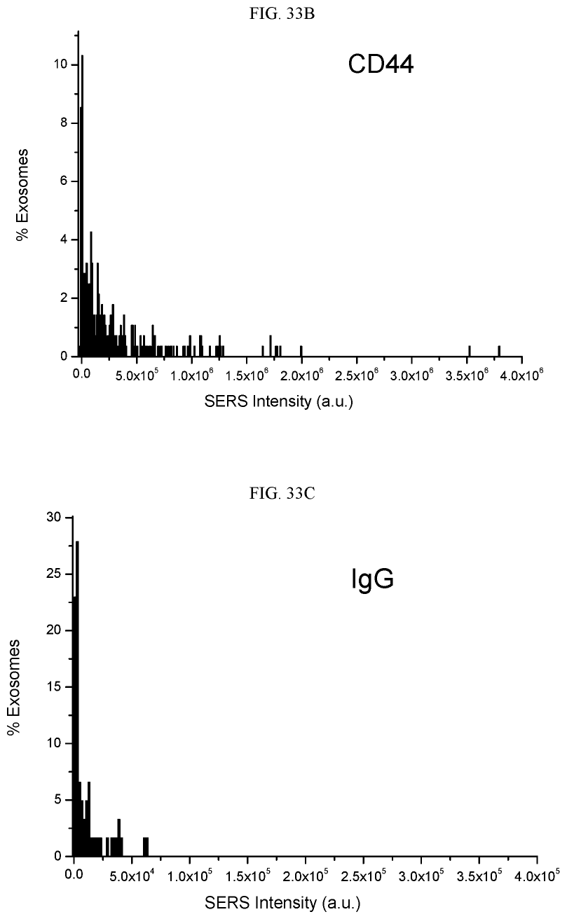

The use of SERS-SVT in single EXO profiling was further demonstrate by analyzing EXOs from a different origin, SKBR3 cells. SKBR3 cells are known to have high expression of HER2 cancer protein markers and low expression of CD44 and thus they represent another good model for technology validation. FIGS. 33A, 33B, and 33C shows the density population profiles of CD44 and HER2 on SKBR3 cells with comparison to IgG control for three different cancer patients. The results show shows that HER2 is highly expressed on SKBR3 EXOs compared to the IgG control and CD44. Using the IgG to define the cut-off value, we calculated that the Fp, ζCD44, and ζCD44, t of the positive HER2 for SKBR3 EXOs is 86%, 4.2×105, and 3.7×105, respectively.

The examples demonstrate that the SERS-SVT method can be used to quantitatively measure protein expressions on EXOs at single exosome level. Measurement of protein level on single exosomes can be used to diagnose cancer potentially at early stages and monitor cancer. Proteins may also be measured on other type of membrane vesicles and used for other type of diseases such as Alzheimer.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A lipophilic substrate comprising an amphiphilic polymer comprising a thiolated hydrophilic portion and a hydrophobic tail covalently bound to a silver or gold film, wherein the film is fixed to a solid support or
comprising 1,2-distearoyl-sn-glycero-3-phosphoethanolamine conjugated polyethylene glycol thiol (DSPE-PEG-SH) and 11-mercaptoundecyl tetra (ethylene glycol) (MU-TEG) covalently bound to a gold film, wherein the film is fixed to a solid support.

2-4. (canceled)

5. The lipophilic substrate of claim 1, wherein the film is gold or silver.

6. An array device comprising
(a) a planar substrate comprising an amphiphilic polymer containing a thiolated hydrophilic portion and a hydrophobic tail covalently bound to a film, wherein the film is fixed to a planar support;
(b) a flexible array interface in contact with the planar substrate, wherein the interface comprises a plurality of holes; and
(c) a rigid array template in contact with the interface, wherein the rigid array comprises a plurality of holes, wherein the holes of the interface and the holes of the array are aligned or
(a) a planar substrate comprising 1,2-distearoyl-sn-glycero-3-phosphoethanolamine conjugated polyethylene glycol thiol (DSPE-PEG-SH) and 11-mercaptoundecyl tetra (ethylene glycol) (MU-TEG) covalently bound to gold film, wherein the film is fixed to the planar substrate;
(b) a flexible array interface in contact with the planar substrate, wherein the interface comprises a plurality of holes; and
(c) a rigid array template in contact with the interface, wherein the rigid array comprises a plurality of holes, wherein the holes of the interface and the holes of the array are aligned.

7. (canceled)

8. The array device of claim 6, wherein the planar substrate is a glass plate or silicon wafer;
wherein the flexible array interface comprises rubber or silicone; and
wherein the rigid array template comprises plastic or resin.

9-12. (canceled)

13. A surface-enhanced Raman scattering nanotag, the nanotag comprising a plasmonic nanoparticle, a 16-mercaptohexadecanoic acid-linked polyethylene glycol covalently bound at the thiol terminal to a surface of the nanoparticle, an antibody bound to the PEG thiol with the thiol terminal bound to a surface of the nanoparticle, and a Raman reporter that is incorporated into the MHDA pocket on the surface of the nanoparticle.

14. The nanotag of claim 10, wherein the Raman reporter is an organic or inorganic dye.

15. The nanotag of claim 13, wherein the organic dye is selected from QSY21, IR820, IR783, BHQ, QXL 680, and DTTC.

16. The nanotag of claim 13, wherein the inorganic dye is pyridine, or aminothiophenol.

17-20. (canceled)

21. The nanotag of claim 13, wherein the Raman reporter that is incorporated into the MHDA pocket is on the surface of a carbon nanosphere or nanotube.

22. (canceled)

23. A surface-enhanced Raman scattering nanotag of claim 13 comprising a plasmonic nanoparticle, a Raman reporter and a cetyltrimethylammonium bromide (CTAB) bilayer.

24-25. (canceled)

26. A method for producing an array device of claim 6, the method comprising
(a) providing a device comprising (a) a planar substrate comprising an amphiphilic polymer containing a thiolated hydrophilic segment and a hydrophobic tail covalently bound to a film, wherein the film is fixed to the planar support;
(b) a flexible array interface in contact with the planar substrate, wherein the interface comprises a plurality of holes; and
(c) a rigid array template in contact with the interface, wherein the rigid array comprises a plurality of holes, wherein the holes of the interface and the holes of the array are aligned, thereby forming a well; and
(b) depositing a target-specific capture molecule into each well of the array, thereby forming a capture array.

27. The method of claim 16, wherein the capture molecule is an antibody, a single-chain antibody, a nanobody, or an aptamer.

28. (canceled)

29. A method for producing an array device of claim 6 comprising a plurality of cells or membrane bound vesicles, the method comprising
(a) providing an array device comprising (i) a planar substrate comprising 1,2-distearoyl-sn-glycero-3-phosphoethanolamine conjugated polyethylene glycol thiol (DSPE-PEG-SH) and 11-mercaptoundecyl tetra (ethylene glycol) (MU-TEG) covalently bound to a gold film in each well, wherein the film is fixed to the planar substrate; (ii) a flexible array interface in contact with the planar substrate, wherein the interface comprises a plurality of holes; and (ii) a rigid array template in contact with the interface, wherein the rigid array comprises a plurality of holes, wherein the holes of the interface and the holes of the array are aligned thereby forming a well; and
(b) depositing into each well of the array device a cell or membrane bound vesicle, thereby forming an array comprising a plurality of cells or membrane bound vesicles.

30. The method of claim 28, wherein the cell is a cancer cell, blood cell, bacterial cell, epithelial cell, or a parasitic cell.

31. The method of claim 19, wherein the membrane bound vesicle is an exosome, microvesicle, an oncosome, microsome, or cellular organelle.

32. An array device comprising a cell or membrane bound vesicle produced according to the method of claim 29.

33. A method for characterizing biomarkers on a plurality of cells or membrane bound vesicles, the method comprising
(a) contacting the array device of claim 32 with a nanotag of claim 13; and
(b) detecting a biomarker present on the cell or membrane bound vesicle using Raman spectroscopy.

34. (canceled)

35. A method for characterizing biomarkers on a plurality of cells or membrane bound vesicles, the method comprising
(a) contacting the array device of claim 6 with a sample comprising a cell or membrane bound vesicle under conditions suitable for binding;
(b) contacting the bound cell or membrane bound vesicle with a nanotag of claim 13; and
(c) detecting a biomarker present on the cell or membrane bound vesicle using Raman spectroscopy.

36. (canceled)

37. A method for characterizing disease in a subject, the method comprising
(a) obtaining a biological sample from the subject, wherein the sample comprises an extracellular vesicle;
(b) contacting a lipophilic substrate of claim 4 with the biological sample under conditions suitable for binding a cell or membrane bound vesicle to the substrate or array device;
(c) contacting the bound extracellular vesicle with a nanotag of claim 13; and
(d) detecting a biomarker present on the cell or membrane bound vesicle using Raman spectroscopy; or
(a) obtaining a biological sample from the subject, wherein the sample comprises an extracellular vesicle;
(b) contacting the array device of claim 4 with the biological sample under conditions suitable for binding the extracellular vesicle to the array device; and
(c) contacting the bound extracellular vesicle with a nanotag of claim 13; and
(d) detecting a biomarker present on the membrane bound vesicle using Raman spectroscopy.

38-39. (canceled)

40. A method for characterizing biomarkers on a membrane bound vesicle, the method comprising:
(a) contacting the membrane bound vesicle with the nanotag of claim 13, wherein an antibody present on the nanotag binds an antigen present on the vesicle;
(b) exposing the membrane bound vesicle to a light source and acquiring an image of the membrane bound vesicle, wherein the image serves as a mask to localize the membrane bound vesicle;
(c) exposing the membrane bound vesicle to a wavelength sufficient to elicit a signal from the nanotag; and
(d) detecting the signal using Raman spectroscopy, thereby characterizing the membrane bound vesicle.

41-43. (canceled)
